(19) United States
(12) Patent Application Publication
(10) Pub. No.: US 2014/0158116 A1
(43) Pub. Date: Jun. 12, 2014

(54) INHIBITING TRPA1 FOR THE TREATMENT OF ASTHMA

(71) Applicant: Cubist Pharmaceuticals, Inc., Lexington, MA (US)

(72) Inventors: Jayhong A. Chong, Cambridge, MA (US); Scott C. Coleman, Lexington, MA (US); Rory Curtis, Lexington, MA (US); Donato del Camino, Cambridge, MA (US); Yu Gui Gu, Lexington, MA (US); Qingyi Li, Lexington, MA (US); Blaise S. Lippa, Lexington, MA (US); Chester A. Metcalf, III, Lexington, MA (US); Magdalene M. Moran, Cambridge, MA (US); Michael D. Ryan, Littleton, MA (US); Dong Zou, Lexington, MA (US)

(73) Assignee: Cubist Pharmaceuticals, Inc., Lexington, MA (US)

(21) Appl. No.: 13/963,021

(22) Filed: Aug. 9, 2013

Related U.S. Application Data
(60) Provisional application No. 61/681,571, filed on Aug. 9, 2012.
(60) Provisional application No. 61/681,506, filed on Aug. 9, 2012.
(60) Provisional application No. 61/798,156, filed on Mar. 15, 2013.

(51) Int. Cl. C07D 473/08; A61M 15/00

(52) U.S. Cl. CPC C07D 473/08; A61M 15/009; A61M 15/0065
USPC 128/200.14; 514/263.21; 128/203.15

(57) ABSTRACT

This disclosure describes novel compounds and pharmaceutical compositions for inhibiting the TRPA1 ion channel and/or medical conditions related to TRPA1, such as asthma.

### RELATED APPLICATIONS

The present application claims priority to U.S. Provisional Application No. 61/681,571, filed Aug. 9, 2012; and claims priority to U.S. Provisional Application No. 61/681,506, filed Aug. 9, 2012, and claims priority to U.S. Provisional Application No. 61/798,156, filed Mar. 15, 2013, all three of which are incorporated herein by reference in their entireties.

### TECHNICAL FIELD

The present disclosure relates to compositions, delivery systems and methods for treating asthma, for example by inhibiting the Transient Receptor Potential A1 ion channel (TRPA1).

### BACKGROUND

Transient Receptor Potential Ion Channel Subfamily A, member 1 (“TRPA1”) is a non-selective cation channel in humans. TRPA1 is found in sensory neurons and functions as a signal transduction receptor linking inflammation to pain. Activation of TRPA1 can increase firing of sensory neurons, leading to the release of pro-inflammatory neuropeptides such as NK-A, substance P and CGRP (which induce vasodilation and help recruit immune cells). A variety of endogenous reactive compounds produced during inflammation activate TRPA1 (including 4-hydroxynonenal released during liposome peroxidation; cyclopentane prostaglandins synthesized by COX enzymes; hydrogen peroxide produced by oxidative stress). TRPA1 can also be activated by a variety of stimuli, including natural products (e.g., allyl isothiocyanate, or AITC), environmental irritants (e.g., acrolein), amphipathic molecules (e.g., trinitrophenol and chlorpromazine) and pharmacological agents. Activation of TRPA1 also sensitizes TRPA1 to cold. Furthermore, a gain-of-function mutation in TRPA1 causes familial episodic pain syndrome; patients suffering from this condition have episodic pain that may be triggered by cold. (Kremeyer et al., Neuron. 2010 Jun. 10; 66(5):671-80).

TRPA1 inhibitor compounds can be used to treat pain. Compounds that inhibit the TRPA1 ion channel can be useful, for example, in treating conditions ameliorated, eliminated or prevented by inhibition of the TRPA1 ion channel (e.g., medical conditions causing pain). Inhibition of TRPA1 (e.g., by genetic ablation and chemical antagonism) has been shown to result in reduced pain behavior in mice and rats. Knockout mice lacking functional TRPA1 have diminished nociceptive responses to TRPA1 activators (including AITC, formalin, acrolein, 4-hydroxynonenal) and, in addition, have greatly reduced thermal and mechanical hypersensitivity in response to the inflammatory mediator bradykinin (e.g., Kwan, K. Y. et al. Neuron 2006, 50, 277-289; Bautista, D. M. et al. Cell 2006, 124, 1269-1282). In animal pain models, down regulation of TRPA1 expression by gene specific antisense oligonucleotides prevented and reversed cold hyperalgesia induced by inflammation and nerve injury (See, e.g., Obata, K. et al., Journal of Clinical Investigation 2005, 115, 2393-2401; Jordt, S. E. et al., Nature 2004, 427, 260-265; Katsura, H. et al., Exploratory Neurology 2006, 200, 112-123). TRPA1 inhibitor compounds are also effective in a variety of rodent pain models. TRPA1 inhibitors have been shown to reduce mechanical hypersensitivity and cold allodynia following inflammation induced by Complete Freund's Adjuvant (without altering normal cold sensation in naïve animals) and also to improve function in the rat mono-iodoacetate osteoarthritis model. (See, del Camino, D. et al. (2010). TRPA1 contributes to cold hypersensitivity. J Neurosci 30, 15165-15174; and Chen, J. et al., (2011). Selective blockade of TRPA1 channel attenuates pathological pain without altering noxious cold sensation or body temperature regulation. Pain 152, 1165-72.) TRPA1 inhibitor compounds have demonstrated reduced pain behavior in rodents injected with AITC (mustard oil), formalin, cinnamaldehyde, acrolein and other TRPA1 activators. (See, Jordt, S. E. et al., Nature 2004, 427, 260-265; Chen, J. et al., (2011).) Selective blockade of TRPA1 channel attenuates pathological pain without altering noxious cold sensation or body temperature regulation. Pain 152, 1165-72.)

Recently, a TRPA1 inhibiting compound was disclosed in PCT patent application PCT/US2009/069146 (published as WO2010/075353A1 on Jul. 1, 2010) (disclosed herein as a compound of Formula (III)):

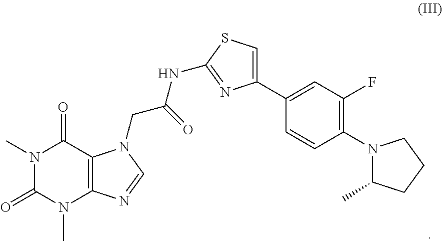 [SMILES: C[C@H]1CCCN1c1ccc(-c2csc(NC(=O)Cn3cnc4c3c(=O)n(C)c(=O)n4C)n2)cc1F]

However, there remains a need to identify compounds that safely modulate (e.g., inhibit) TRPA1 ion channels. In particular, there is a need to identify compounds that inhibit TRPA1 with reduced levels serum biomarkers of hepatotoxicity than observed upon administration of the compound of Formula (III) above in certain animal models (see, e.g., Example 8 herein). Such compounds are useful, for example, both as research tools and as therapeutic agents (e.g., for the treatment of pain).

The compound of Formula (I) is another antagonist of the human and animal TRPA1 channel that demonstrated reduced levels of serum biomarkers of hepatotoxicity compared to the compound of Formula (III) (see, e.g., Example 8). Stereoisomers and deuterated compounds of Formula (I) can be made according to Examples 1A-1C. Formula (I) is disclosed in the PCT patent application PCT/US2012/050210, filed Aug. 9, 2012 (published as WO 2013/023102 on Feb. 14, 2013).

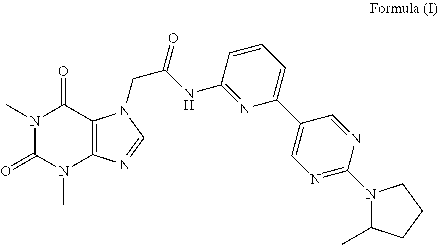 [SMILES: CC1CCCN1c1ncc(-c2cccc(NC(=O)Cn3cnc4c3c(=O)n(C)c(=O)n4C)n2)cn1]

Compounds of Formula (I) include the compound of Formula (Ia), a first stereoisomer of Formula (I) that can be synthesized according to the synthesis of FIG. 1A, as described in Example 1A, and as a pharmaceutically acceptable salt (e.g., a hydrochloride salt described in Example 2).

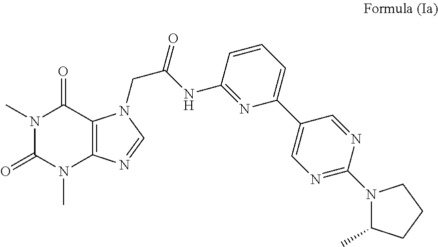 [SMILES: C[C@H]1CCCN1c1ncc(-c2cccc(NC(=O)Cn3cnc4c3c(=O)n(C)c(=O)n4C)n2)cn1]

The compound of Formula (Ib) is a second stereoisomer of Formula (I) that can be synthesized according Example 1c, and as a pharmaceutically acceptable salt.

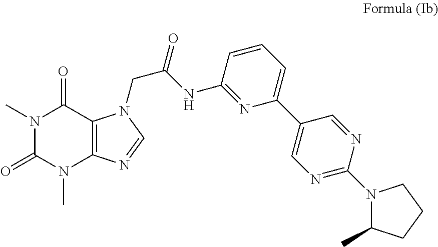 [SMILES: C[C@@H]1CCCN1c1ncc(-c2cccc(NC(=O)Cn3cnc4c3c(=O)n(C)c(=O)n4C)n2)cn1]

The compound of Formula (Ib) is a second stereoisomer of Formula (I), and is a small molecule antagonist of the human TRPA1 channel in in vitro testing.

### SUMMARY

Novel methods of treating asthma can include the administration of a therapeutically effective amount of a compound of Formula (I). A compound of Formula (I) was evaluated in an allergic asthma animal model (e.g., Example 12). The discovery of the new methods of treating asthma or asthma symptoms are based in part on measurements of the effects of different doses of the compound of Formula (Ia) on pulmonary resistance (FIG. 9A), quantification of the effects of different doses of the compound of Formula (Ia) during the late response of pulmonary resistance (FIG. 9B), and the effects of different doses of the c of the compound of Formula (Ia) on airway hyper-responsiveness (FIG. 10). Pharmaceutical compositions comprising a compound of Formula (I) (e.g., a compound of Formula (Ia)) are useful for administration for the treatment of asthma.

Other pharmaceutical compositions for treating asthma can include a compound of Formula (I) containing compounds of Formula (Ia) and/or Formula (Ib). The pharmaceutical compositions comprising the compound(s) of Formula (I) (e.g., compounds of Formula (Ia) and/or Formula (Ib)) are useful in the manufacture of pharmaceutical compositions for treating asthma. A compound of Formula (I) (e.g., Formula (Ia) and/or Formula (Ib)) is also useful in the manufacturing of pharmaceutical compositions for treating a respiratory condition such as asthma, preferably a condition responsive to a TRPA1 inhibitor. For example, pharmaceutical compositions comprising a compound of Formula (I) can be administered by an intranasal or inhaled route of delivery.

A compound of Formula (I) or a pharmaceutically acceptable salt thereof can be used in the manufacture of a pharmaceutical composition for treating asthma. For example, the compound of Formula (Ia) can be in the form of a hydrochloride salt, formulated as an aerosol, and/or administered by inhalation. The pharmaceutical composition can be indicated for the treatment of human asthma, such as human asthma selected from the group consisting of: cold induced asthma, exercise-induced asthma, allergy-induced asthma, and occupational asthma. The pharmaceutical composition can include and/or be administered as a dose of about 0.5-25 mg/kg, or 5-10 mg/kg. The pharmaceutical composition can be formulated with a pH in the range of about 4.5 to 5.5. A unit dosage form can include a compound of Formula (I) (e.g., a compound of Formula (Ia)) in an amount of about 200 mg-1600 mg, such as a unit dosage form that comprises about 200 mg, 400 mg, 800 mg, 1200 mg or 1600 mg of a compound of Formula (I) (e.g., a compound of Formula (Ia)). The pharmaceutical composition can be formulated and/or delivered as droplets of about 5 mm or less in diameter. Pharmaceutical compositions comprising a compound of Formula (I) can be packaged in a metered dose aerosol dispenser containing the pharmaceutical composition, such as a metered dose inhaler, a dry powder inhaler or an air-jet nebulizer.

In one example, a compound of Formula (Ia) can be used for treating asthma in the manufacture of a pharmaceutical composition formulated for inhalation or intranasal administration of a therapeutically effective amount of a compound of Formula (Ia), or a pharmaceutically acceptable salt thereof. The pharmaceutical composition can be administered in a unit dosage form that comprises about 200 mg-1600 mg of a compound of Formula (Ia), where the compound of Formula (Ia) can be in the form of a hydrochloride salt. The compound of Formula (Ia) can be administered by inhalation or by intranasal administration.

A metered dose aerosol dispenser can contain a pharmaceutical composition adapted for pulmonary or nasal delivery, where the pharmaceutical composition comprising a compound of Formula (Ia), or a pharmaceutically acceptable salt thereof.

### BRIEF DESCRIPTION OF THE DRAWINGS

FIG. 1A is an exemplary reaction scheme to synthesize a compound of Formula (Ia), as described in Example 1A.

FIG. 1B is a reaction scheme to synthesize a deuterated compound (12), a deuterated analog of the compound of Formula (Ia), as described in Example 1B.

FIG. 2 is a bar graph demonstrating the effect of administering a pharmaceutical composition comprising the compound of Formula (Ia) at different concentrations (3, 10, 30, and 50 mg/kg) to rodents prior to conducting a formalin injection as described in Example 5. FIG. 2 shows the measured pain duration (as the number (n) of seconds over a 2 minute observation period) in a rodent formalin injection pain model for various pharmaceutical compositions containing different amounts of the compound of Formula (Ia), a vehicle delivered intraperitoneally (i.p.), and the comparator compound of Formula (II).

FIG. 3 is a line graph demonstrating increased Paw Withdrawal Latency (PWL) scores observed after i.p. administration of pharmaceutical compositions with increasing concentrations of the compound of Formula (Ia) in the Complete Freund's Adjuvant (CFA) rodent model described in Example 6. FIG. 3 shows the change in PWL score as a function of the concentration of the compound of Formula (Ia), as well as the PWL scores observed upon administration of the vehicle alone and a comparator pharmaceutical composition containing the comparator compound of Formula (II).

FIG. 4 is a line graph demonstrating reduction in guarding scores observed after i.p. administration of pharmaceutical compositions with various concentrations of the compound of Formula (Ia) in the rodent incisional pain model described in Example 7. FIG. 4 shows the change in guarding score as a function of the administered concentration of the compound of Formula (Ia), as well as the guarding scores observed upon administration of the vehicle alone and comparator pharmaceutical compositions containing the comparator compound of Formula (III) or ketoprofen.

FIG. 5A is a bar graph of data for measurement of serum chemistry biomarkers of hepatotoxicity measured in female dogs orally dosed with a compound of Formula (Ia). The figures along the X-axis are the doses of compound of Formula (I) administered.

FIG. 5B is a bar graph of data for measurement of serum chemistry biomarkers of hepatotoxicity measured in male and female dogs orally dosed with a comparator compound of Formula (III).

FIG. 6 is a bar graph of data showing the effect on hepatotoxicity biomarkers in rat serum for administering a compound of Formula (Ia) or a comparator compound of Formula (III) in a 7-day i.p. repeat dose screening toxicity study at 50 mg/kg/day for 7 consecutive days.

FIG. 7 is the characteristic NMR spectrum identifying the compound of Formula (Ib).

FIG. 8 is a flow chart showing the steps in the experimental protocol for the evaluation of the compound of Formula (Ia) in an allergic asthma model.

FIG. 9A is a line graph of data showing the effects of different doses of the compound of Formula (Ia) on pulmonary resistance.

FIG. 9B is a bar graph of data showing the quantification of the effects of different doses of the compound of Formula (Ia) during the late response of pulmonary resistance.

FIG. 10 is a bar graph of data showing the effects of different doses of the c of the compound of Formula (Ia) on airway hyper-responsiveness.

### DETAILED DESCRIPTION

A compound of Formula (I) (e.g., Formula (Ia)) and pharmaceutically acceptable salts thereof, are useful for the inhibition of the TRPA1 ion channel in pharmaceutical compositions as well as research tools.

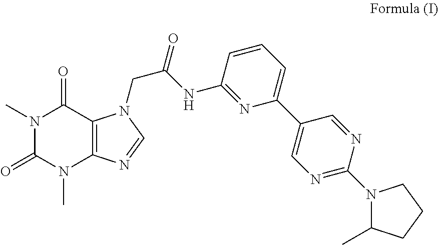 [SMILES: CC1CCCN1c1ncc(-c2cccc(NC(=O)Cn3cnc4c3c(=O)n(C)c(=O)n4C)n2)cn1]

### Inhibiting TRPA1 with the Compound of Formula (Ia)

The compound of Formula (Ia) is a small molecule antagonist of the human TRPA1 channel in both in vitro and in vivo testing. The compound of Formula (Ia) is also a highly selective in vitro inhibitor of TRPA1. For example, the compound of Formula (Ia) blocks inward currents through TRPA1 in rat, dog and humanTRPA1 (Example 3). The antagonist effect of the compound of Formula (Ia) against human TRPA1 (hTRPA1) was measured in a whole cell patch configuration (Example 3). Furthermore, the compound of Formula (Ia) is highly selective for TRPA1 as compared with known TRP channels and voltage-gated ion channels (Example 3). The compound of Formula (Ia) can be used in assays for identifying compounds that inhibit TRPA1. A compound of Formula (I) can also be used in a method of modulating a TRPA1 ion channel, comprising contacting a cell with a compound having the structure of Formula I (e.g., a compound of Formula (Ia)), or a pharmaceutically acceptable salt thereof.

The compound of Formula (Ia) is an active pharmaceutical compound in multiple in vivo rat models of pain, including pain induced by direct activation of the TRPA1 channel with formalin injection (Example 5), cold allodynia following chronic Complete Freund's Adjuvant-induced inflammation (Example 6), and a rodent surgical model involving the incision of the plantar surface of the hind paw (Example 7).

The compound of Formula (Ia) is a novel small molecule antagonist of the TRPA1 channel as demonstrated by in vitro testing. The compound of Formula (Ia) and blocks inward currents through TRPA1 in rat, dog and human with an IC₅₀ of approximately 100 nanomolar (Table 1, data obtained according to Example 3). The antagonist effect of the compound of Formula (Ia) against hTRPA1 in a whole cell patch configuration was evaluated according to the method of Example 3.

TABLE 1

| | | | TESTED | | IC₅₀ |
|---|---|---|---|---|---|
| | | | CONCS. | CURRENT | Inward |
| CHAN- | SPE- | COM- | (nano- | ACTI- | current |
| NEL | CIES | POUND | molar) | VATION | (nanomolar) |
| hTRPA1 | Human | Formula | 10, 32, | 10 mi- | 93 ± 22 |
| | | (Ia) | 100, 320, | cromolar | |
| | | | 1000 | AITC | |
| rTRPA1 | Rat | Formula | 32, 100, | 10 mi- | 101 ± 8 |
| | | (Ia) | 320, 1000, | cromolar | |
| | | | 3200 | AITC | |
| dTRPA1 | Dog | Formula | 32, 100, | 10 mi- | 102 ± 20 |
| | | (Ia) | 320, 1000 | cromolar | |
| | | | | AITC | |

The compound of Formula (Ia) is highly selective for hTRPA1 as compared with TRP channels and voltage-gated ion channels. For example, when tested against eight different channels representing most of the ion channel families (Table 2, Example 3), none of the tested channels was reproducibly blocked or agonized by the compound of Formula (Ia) at physiologically relevant concentrations (e.g., 1, 3.2, 10, or 32 micromolar). Because the highest concentrations used (32 micromolar) had little effect, the actual IC₅₀ of the compound of Formula (Ia) for most of these channels cannot be determined. However, the compound of Formula (Ia) is at least 100-fold selective for block of TRPA1 over all other tested channels (Table 2, Example 3).

TABLE 2

| | | | | | Fold |
|---|---|---|---|---|---|
| | TESTED | | | | Selectivity |
| | CONCS. | CURRENT | CURRENT | | Compared to |
| CHANNEL | (μM) | ACTIVATION | EVALUATED | IC₅₀ (μM) | TRPA1 |
| hTRPV1 | 1, 10 | 500 nanomolar | Inward (−80 mV) | >10 | >100 |
| | | Capsaicin | | | |
| hTRPV3 | 1, 3.2, 10, | 30 micromolar | Inward (−80 mV) | >32 | >300 |
| | 32 | 2-APB | | | |
| hTRPV4 | 3.2, 10, 32 | 2 micromolar | Inward (−80 mV) | 16 | ~170 |
| | | 4α-PDD | | | |
| hTRPV4 | 3.2, 10, 32 | None | Inward (−80 mV) | No Effect | N/A |
| Agonist | | | | | |
| hTRPV6 | 1, 3.2, 10, | Voltage | Inward (−80 mV) | 34 | ~370 |
| | 32 | | | | |
| hTRPC5 | 1, 10 | 80 micromolar | Inward (−80 mV) | >10 | >100 |
| | | LaCl₃ | | | |
| hTRPM8 | 1, 3.2, 10, | 100 micromolar | Inward (−80 mV) | 19 | ~200 |
| | 32 | Menthol | | | |
| hERG | 1, 10 | Voltage | Tail current (−40 mV) | >10 | >100 |
| hNa_{V}1.2 | 1, 3, 10 | Voltage | Peak Inward (0 mV) | >10 | >100 |

The compound of Formula (Ia) is a novel small molecule antagonist of the human TRPA1 channel as demonstrated by in vivo testing. For example, the compound of Formula (Ia) was active in rodent models of pain in vivo induced by the TRPA1 channel with formalin injection.

The in vivo activity of the compound of Formula (Ia) can be compared to the activity of comparator compounds of Formula (II), Formula (III), and Formula (IV).

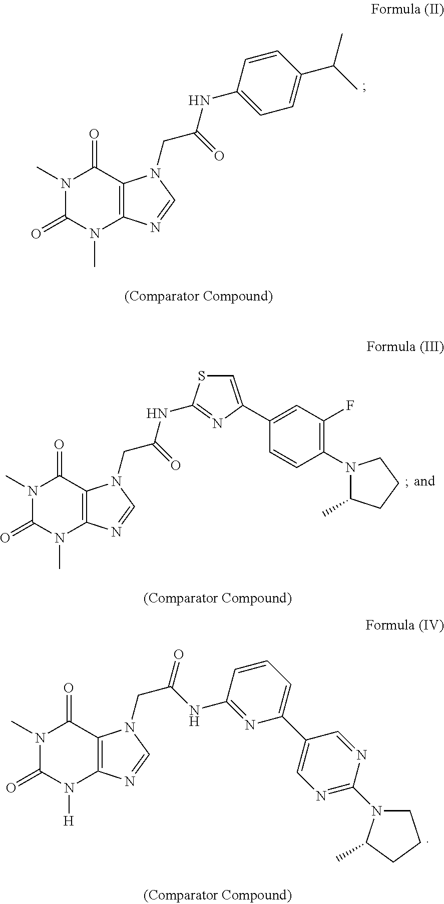

The compound of Formula (II) is a known TRPA1 inhibitor (see, e.g., U.S. Pat. No. 7,671,061) and was therefore used as a positive control. The compound of Formula (II) and methods of making and using this compound are disclosed as the TRPA1 inhibitor compound 200 in U.S. Pat. No. 7,671,061 (filed Dec. 22, 2006, issued Mar. 2, 2010).

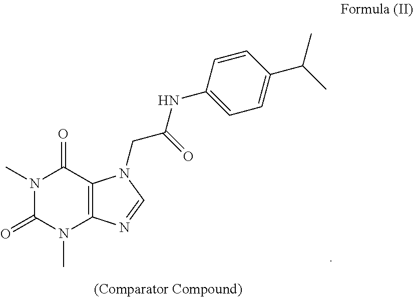 [SMILES: CC(C)c1ccc(NC(=O)Cn2cnc3c2c(=O)n(C)c(=O)n3C)cc1]

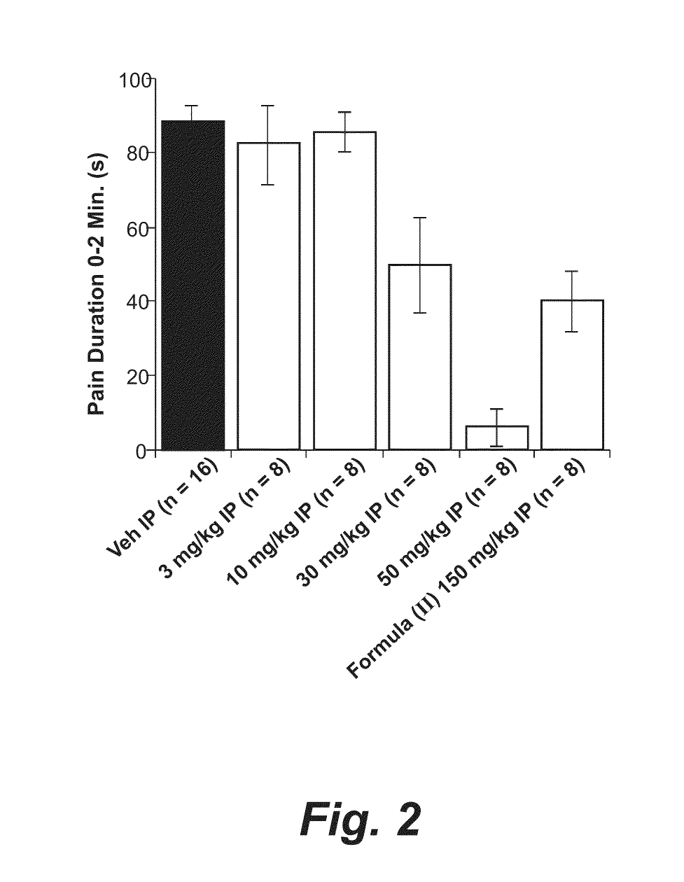

The data shown in Tables 3a, 3b, and 3c and FIG. 2 were obtained by administering a pharmaceutical composition comprising the compound of Formula (Ia) to rodents in the formalin-induced pain duration at various doses according to Example 5. Specifically, the data in Tables 3a, 3b, and 3c and FIG. 2 were obtained by intraperitoneal (i.p.) administration of compositions containing different concentrations of the compound of Formula (Ia), a comparator composition containing recited amounts of the comparator compound (e.g., 150 mg/kg of the comparator compound of Formula (II) in Table 3a) and a control composition containing the vehicle (e.g., without the compound of Formula (Ia) or a comparator compound). As shown in Tables 3a, 3b, and 3c and FIG. 2, the animals treated with the compounds of Formulae (Ia), (II), and (III) showed shorter durations of pain behavior than those treated with the vehicle. The data demonstrates that the compound of Formula (Ia) has an analgesic effect on pain caused by TRPA1 activation with formalin.

TABLE 3a

| | Duration of Pain Behavior | Error |
|---|---|---|
| Compound and Dose | (seconds) | (seconds) |
| Vehicle | 88.6 | 4.3 |
| 3 mg/kg Formula (Ia) | 82.3 | 10.6 |
| 10 mg/kg Formula (Ia) | 85.8 | 5.4 |
| 30 mg/kg Formula (Ia) | 49.8 | 12.8 |
| 50 mg/kg Formula (Ia) | 5.9 | 5.0 |
| 150 mg/kg Formula (II) | 40.0 | 8.1 |

TABLE 3b

| | Duration of Pain Behavior | Error |
|---|---|---|
| | (seconds) | (seconds) |
| 50 mg/kg Formula (III) | 44.3 | 10.5 |
| Vehicle | 77.2 | 3.6 |

TABLE 3c

| | # of Flinches | Error |
|---|---|---|
| 300 mg/kg Formula (II) | 43 | 9 |
| 100 mg/kg Formula (II) | 62 | 17 |
| 30 mg/kg Formula (II) | 88 | 19 |
| Vehicle | 120 | 17 |
| Gabapentin (reference) | 75 | 13 |

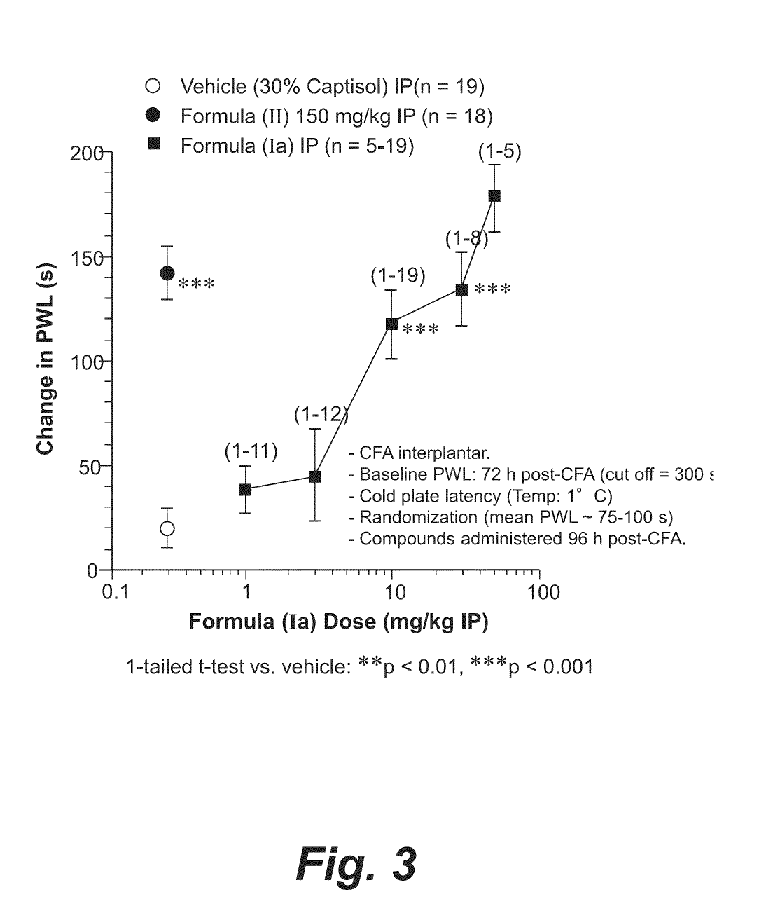

The compound of Formula (Ia) is also active in rodent models of pain in vivo induced by cold allodynia following chronic Complete Freund's Adjuvant-induced inflammation, as described in Example 6. The data presented in Table 4 and FIG. 3 demonstrate increased Paw Withdrawal Latency (PWL) scores observed after i.p. administration of pharmaceutical compositions with increasing concentrations of the compound of Formula (Ia) in the Complete Freund's Adjuvant (CFA) rodent model described in Example 6. This data was obtained by measuring the change in PWL score as a function of the concentration of the compound of Formula (Ia), as well as the PWL scores observed upon administration of a composition containing the comparator compound of Formula (II) and a control with the vehicle containing a sulfobutylether β-cyclodextrin compound (available under the trade name Captisol® from CyDex Pharmaceuticals, Inc, Lenexa, Kans.). The data shows that the compound of Formula (Ia) has an analgesic effect on cold allodynia.

TABLE 4

| | Change in Paw Withdrawal | |
|---|---|---|
| Compound and Dose | Latency | Error |
| Vehicle | 19.8 | 9.4 |
| 1 mg/kg Formula (Ia) | 38.4 | 11.5 |
| 5 mg/kg Formula (Ia) | 45.0 | 22.0 |
| 10 mg/kg Formula (Ia) | 117.6 | 16.6 |
| 30 mg/kg Formula (Ia) | 134.4 | 17.8 |
| 50 mg/kg/ Formula (Ia) | 177.8 | 15.5 |
| 150 mg/kg Formula (II) | 142.2 | 12.3 |

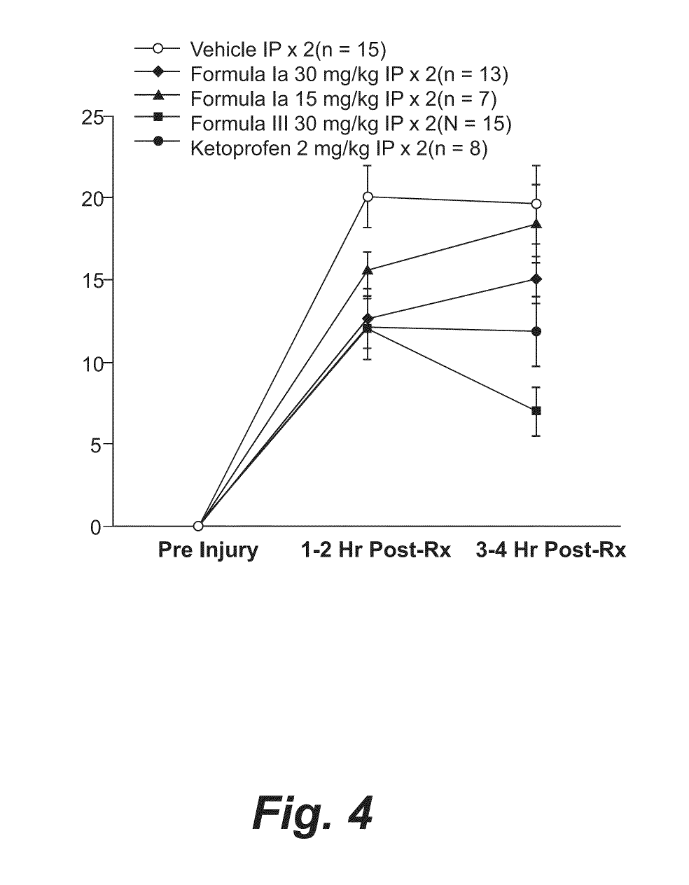

The compound of Formula (Ia) is also active in rodent models of pain in vivo induced by incision of the plantar surface of the hind paw (i.e., the “Brennan Surgical Model”), as described in Example 7. FIG. 4 shows the change in guarding score as a function of the administered concentration of the compound of Formula (Ia), as well as the guarding scores observed upon administration of the vehicle alone and comparator pharmaceutical compositions containing the comparator compound of Formula (III), or ketoprofen. Referring to FIG. 4 and Example 7, 60 mg/kg of the compound of Formula (Ia) delivered intraperitoneally (2 doses of 30 mg/kg before and immediately after the surgery) reduces spontaneous pain in the rodent incisional pain model described in Example 7 for up to 4 hours after surgery, better than ketoprofen (2 doses of 2 mg/kg intraperitoneally). Thirty (30) mg/kg of the compound of Formula (Ia) delivered intraperitoneally (2 doses of 15 mg/kg before and immediately after the surgery) reduces spontaneous pain for up to 2 hours after surgery (FIG. 4).

A comparator TRPA1 inhibitor of Formula (III) was also tested in the Brennan rodent model of Example 7 (FIG. 4). The comparator compound of Formula (III) and methods of making and using this compound are disclosed as the TRPA1 inhibitor compound I in PCT patent application PCT/US2009/069146 (published as WO2010/075353A1 on Jul. 1, 2010).

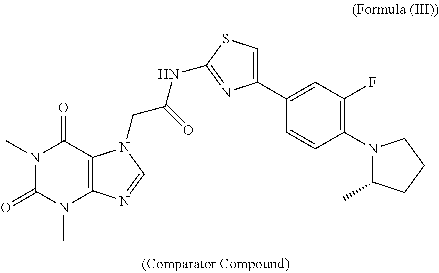 [SMILES: C[C@H]1CCCN1c1ccc(-c2csc(NC(=O)Cn3cnc4c3c(=O)n(C)c(=O)n4C)n2)cc1F]

The in vitro TRPA1 activity of a comparator compound of Formula (IV) was measured:

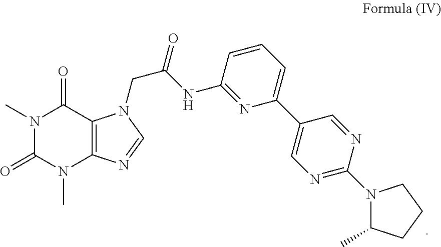 [SMILES: C[C@H]1CCCN1c1ncc(-c2cccc(NC(=O)Cn3cnc4c3c(=O)n(C)c(=O)n4C)n2)cn1]

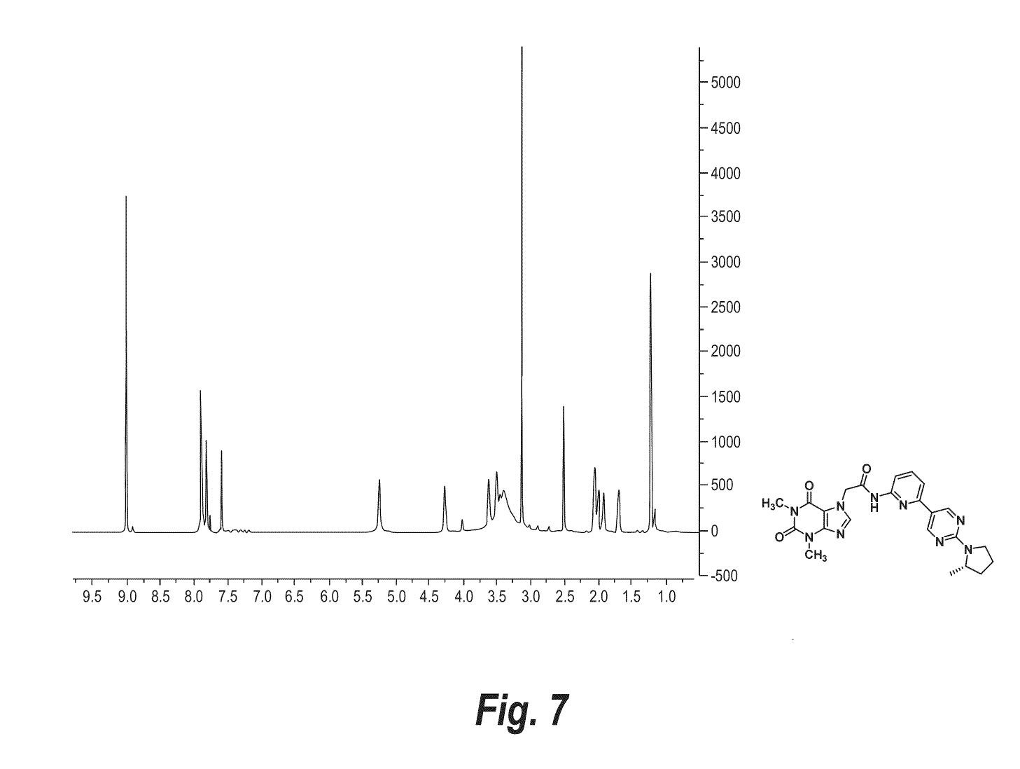

The chemical structure of the comparator compound of Formula (IV) was identified using nuclear magnetic resonance NMR. The NMR sample was prepared by dissolving approximately 1.85 mg of the metabolite of Formula (Ia) in 50 μL of NMR solvent. The sample was bath sonicated for 1 min to ensure proper dissolution before it was pipette into the NMR tube. The tube was sealed with a plastic ball and stored at room temperature prior to the experiments. NMR experiments were performed on a 600 MHz Bruker Avance III NMR Spectrometer equipped with a 1.7 nM Cryo-TCI probe. The sample was inserted into the magnet using a SampleJet accessory. In order to obtain complete connectivities for this molecule, a standard ¹H-NMR spectrum, a multiplicity-edited ¹H-¹³C gHSQC spectrum, and a ¹H-¹³ gHMBC spectrum were recorded (FIG. 7). The comparator compound of Formula (IV) has a TrpA1 IC50 of 9.8 μM, and in vitro selectivity characterized by: TrpV3>10 μM, hERG>20 μM, NaV1.2>20 μM (Table 5). This data was collected using the same procedure as that of Example 3.

TABLE 5
IC₅₀ in Patch Clamp Assay

| Assay | IC50 | |
|---|---|---|
| TRPA1 | 9.8 | μM |
| TrpV3 | >10 | μM |
| hERG | >20 | μM |
| NaV1.2 | >20 | μM |

The compounds disclosed herein (e.g., a compound of Formula (I) or Formula (Ia)) can be used in assays for identifying compounds that inhibit TRPA1. For example, a method of identifying a TRPA1 inhibitor can include the steps of: contacting a test compound with a TRPA1 ion channel, measuring the inhibition of the TRPA1 ion channel by the test compound (e.g., generating a first IC50 value for the test compound), comparing the measurement of TRPA1 ion channel inhibition by the test compound with a second measurement of a second TRPA1 ion channel after contact with the compound of Formula (I) (e.g., measuring a second IC₅₀ value for the compound of Formula (I) or Formula (Ia)), and determining whether the test compound is a TRPA1 inhibitor by comparison of the first and second measurements of TRPA1 ion channel inhibition. The TRPA1 ion channel inhibition by the compound of Formula (I) (e.g., Formula (Ia)) (or compounds of Formula (II), (III), or (IV)) can be used as a comparator to the test compound. The measurement of TRPA1 ion channel inhibition can be performed by any suitable assay, including the assay of Example 3 (e.g., patch clamp protocol). In one embodiment, a method for identifying a TRPA1 ion channel inhibitor compound comprises contacting a TRPA1 protein in a cell-based assay with a test agent to be tested for potential activity as a TRPA1 inhibitor; determining whether the test agent increases or decreases the activity of the TRPA1 protein; selecting the agent that decreases the activity of the TRPA1 protein; determining the degree of TRPA1 inhibition of said agent that decreases the activity of the TRPA1 protein; and comparing the degree of TRPA1 inhibition of said agent that decreases the activity of the TRPA1 protein relative to the degree of TRPA1 inhibition observed by a reference agent, whereby a decrease in the degree of TRPA1 inhibition of said agent relative to the degree of TRPA1 inhibition by the reference agent identifies said test agent as a TRPA1 inhibitor. The reference agent can be (for example) a compound of Formula (Ia), (II), (III), or (IV).

The compound of Formula (Ib) is a second stereoisomer of Formula (I).

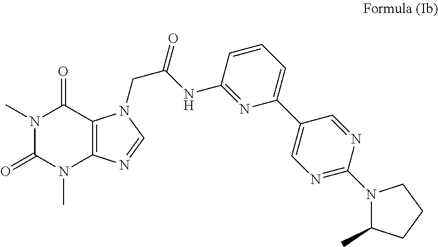 [SMILES: C[C@@H]1CCCN1c1ncc(-c2cccc(NC(=O)Cn3cnc4c3c(=O)n(C)c(=O)n4C)n2)cn1]

The compound of Formula (Ib) can be synthesized according Example 1c, and as a pharmaceutically acceptable salt. The compound of Formula (Ib) is a novel small molecule antagonist of the human TRPA1 channel in in vitro testing. The in vitro TRPA1 activity of the compound of Formula (Ib) shown below was measured, having an IC₅₀ against hTRPA1 of between 50 and 100 nM as provided in Table 6 below.

TABLE 6

| | | | TESTED | | IC₅₀ |
|---|---|---|---|---|---|
| | | | CONCS. | CURRENT | Inward |
| CHAN- | SPE- | COM- | (nano- | ACTI- | current |
| NEL | CIES | POUND | molar) | VATION | (nanomolar) |
| hTRPA1 | Human | Formula | 10, 32, | 10 mi- | 77 |
| | | (Ib) | 100, 320, | cromolar | |
| | | | 1000 | AITC | |

In addition, the IC₅₀ for hTrpA1 measured in 1% RSA (rat serum albumin) was 15.2 μM for the compound of Formula (Ib), compared to 5.3 μM for the compound of Formula (Ia).

### Synthesis of the Compound of Formula (I) and Salts Thereof

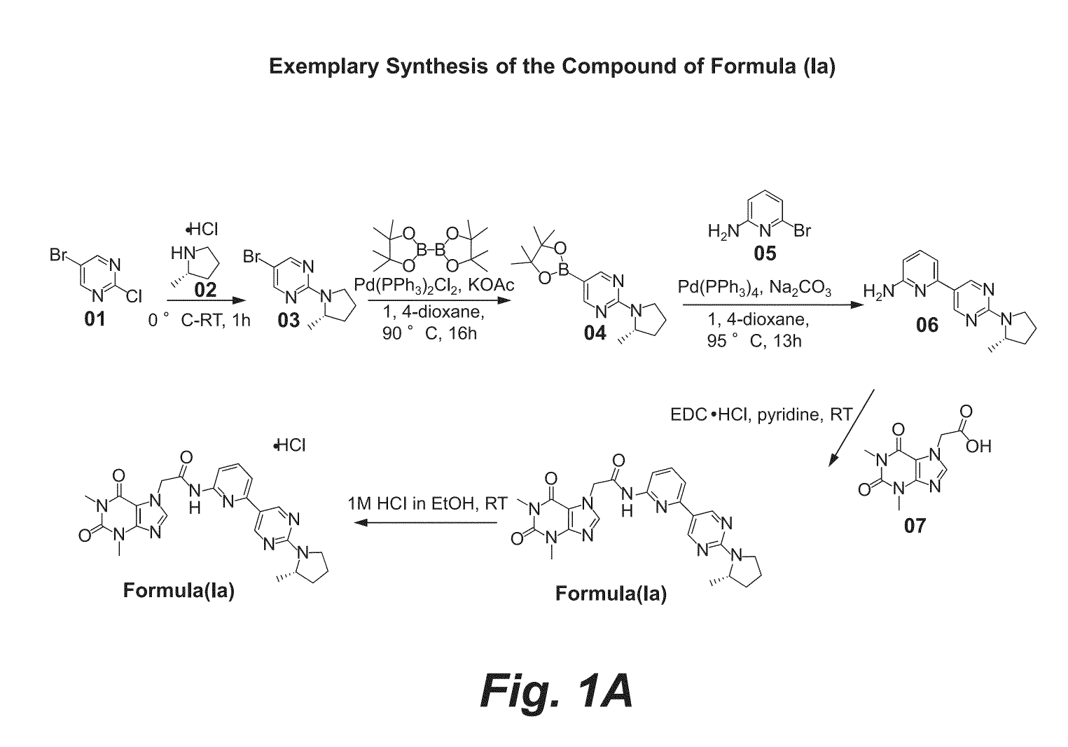

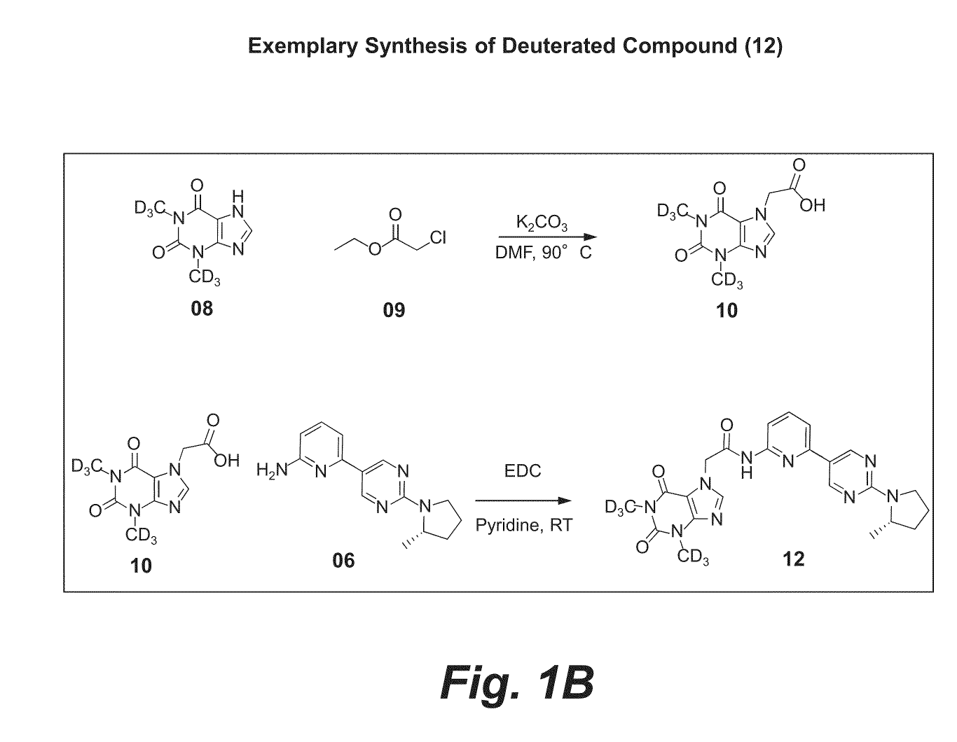

The compound of Formula (Ia) is a stereoisomer of Formula (I) that can be made by multi-step synthetic processes shown in FIG. 1A, as described in Example 1A.

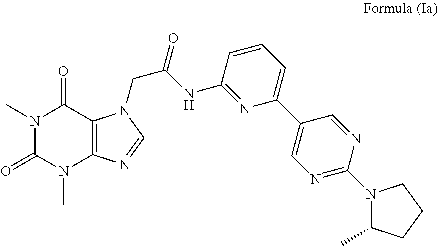 [SMILES: C[C@H]1CCCN1c1ncc(-c2cccc(NC(=O)Cn3cnc4c3c(=O)n(C)c(=O)n4C)n2)cn1]

Briefly, referring to FIG. 1A, the compound of Formula (Ia) can be formed by: (1) reacting (S)-2-methylpyrrolidine 02 with 5-bromo-2-chloropyrimidine 01 to form the intermediate compound 03, (2) coupling the compound 03 intermediate with compound 05 (6-bromo-2-aminopyridine) by one or more reactions to form the intermediate compound 06, and (3) reacting compound 06 with compound 07 in a coupling reaction to form the compound of Formula (Ia). While coupling of the compound 03 intermediate with compound 05 can be performed via the intermediate compound 04, as shown in FIG. 1A and described in Example 1A, other synthetic schemes are also suitable for preparation of the compound of Formula (Ia). As described in Example 1A and FIG. 1A, the intermediate compound 06 can be formed by reacting compound 03 with bis(pinacolato)diboron and other materials to form the intermediate compound 04, followed by reaction of the intermediate compound 04 with 6-bromo-2-aminopyridine (compound 05) to obtain the intermediate compound 06. Each of the reaction steps can be performed with suitable reagents with reaction conditions suitable for obtaining the product(s) indicated in FIG. 1A.

Optionally, the process for synthesizing the compound of Formula (Ia) can further include steps for isolating the intermediate compounds 03 and compound 06 prior to performing subsequent reactions. In addition, the compound of Formula (Ia) can optionally be converted to a pharmaceutically acceptable salt. In FIG. 1A, the conversion of the compound of Formula (I) to a pharmaceutically acceptable HCl salt of a compound of Formula (Ia) is shown according to Example 2.

The compound of Formula (Ib) is a second stereoisomer of Formula I.

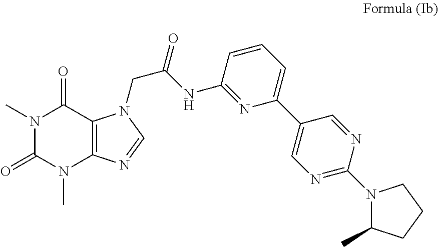 [SMILES: C[C@@H]1CCCN1c1ncc(-c2cccc(NC(=O)Cn3cnc4c3c(=O)n(C)c(=O)n4C)n2)cn1]

The compound of Formula (Ib) can be synthesized using a similar procedure as described above for making the compound of Formula (Ia), by replacing (S)-2-methylpyrrolidine with (R)-2-methylpyrrolidine (i.e., substitution of compound 02 in FIG. 1A with (R)-2-methylpyrrolidine). A racemic compound of formula (I) can also be prepared, for example, by using a racemic 2-methylpyrrolidine instead of compound 02 in the reaction scheme in FIG. 1A, or by combining a compound of Formula (Ia) with a compound of Formula (Ib). Compositions of Formula (I) comprising over 95% enantiomeric excess of the compound of Formula (Ia) over Formula (Ib) can be made by selecting (S)-2-methylpyrrolidine starting material with sufficient enantiomeric purity (i.e., greater than 95%). Similarly, compositions of Formula (I) comprising over 95% enantiomeric excess of the compound of Formula (Ib) over Formula (Ia) can be made by selecting (R)-2-methylpyrrolidine starting material with sufficient enantiomeric purity (i.e., greater than 95%). Compositions of Formula (I) having desired amounts of both stereoisomers of Formula (Ia) and Formula (Ib) can be made by combining pre-determined amounts of compositions of Formula (Ia) with greater than 95% enantiomeric purity with compositions of Formula (Ib) with greater than 95% enantiomeric purity, each made with 2-methylpyrrolidine starting material with the corresponding stereochemistry.

The term “enantiomeric excess” a number from 0 to 100, zero being racemic and 100 being pure, single enantiomer. A compound which in the past might have been called 98% optically pure is now more precisely described as 96% e.e.; in other words, a 90% e.e. reflects the presence of 95% of one enantiomer and 5% of the other in the material in question. A compound of Formula (I) can be obtained as a pharmaceutically acceptable salt.

The term, “pharmaceutically acceptable salts” of the compound of Formula (I) (e.g., Formula (Ia)), refers to salts prepared from pharmaceutically acceptable non-toxic acids including inorganic acids and organic acids. One particularly preferred salt form of the compound of Formula (I) (e.g., Formula (Ia)) is the hydrochloride salt disclosed in Example 2. In general, pharmaceutically acceptable salts of Formula (I) (e.g., Formula (Ia)) can be prepared to improve stability or toxicological properties of the compound, increase or decrease solubility, wettability, improve pharmacokinetic performance of the compound (e.g., Cₘₐₓ or AUC measurements) or improve storage properties (e.g., to reduce hygroscopicity) of a pharmaceutical composition.

### Pharmaceutical Compositions Comprising the Compound of Formula (I)

The compound of Formula (I) (e.g., a compound of Formula (Ia)) or a pharmaceutically acceptable salt thereof can be used in the manufacture of pharmaceutical compositions. Pharmaceutical compositions can be formed by combining the compound of Formula (I) (e.g., a compound of Formula (Ia)), or a pharmaceutically-acceptable salt thereof, with a pharmaceutically acceptable carrier. The pharmaceutical composition can be formulated with a pharmaceutically-acceptable carrier suitable for delivery to a recipient subject (e.g., a human) in accordance with a desired method of drug delivery. Pharmaceutical compositions, particularly those formulated for oral delivery, preferably comprise the compound of Formula (I) (e.g., a compound of Formula (Ia)), or a salt of the compound of Formula (I) (e.g., a compound of Formula (Ia)), in an amount sufficient to achieve the intended purpose (e.g., the treatment or prevention of pain or other conditions responsive to inhibition or antagonism of the TRPA1 ion channel) and one or more additional carriers such as solubilizing agents (e.g., cyclodextrin and/or cyclodextrin derivatives), buffering agents, preservatives and the like (see, e.g., Example 10). The amount and concentration of compound of Formula (I) (e.g., a compound of Formula (Ia)) in the pharmaceutical composition, as well as the quantity of the pharmaceutical composition administered to a subject, can be selected based on clinically relevant factors, such as medically relevant characteristics of the subject (e.g., age, weight, gender, other medical conditions, and the like), the solubility of the compound in the pharmaceutical composition, the potency and activity of the compound, and the manner of administration of the pharmaceutical composition. For example, a pharmaceutical composition can be formulated for oral delivery of the compound of Formula (I) dissolved in a clinically-tolerated amount of a hydroxypropyl-beta-cyclodextrin (e.g., Formula (Ia) as shown in Example 10).

Pharmaceutical compositions may be formulated for a suitable route of administration for providing the patient with an effective dosage of a compound of the present invention. For example, oral administration may be employed (e.g., swallowed or inhaled). Dosage forms include tablets, troches, dispersions, suspensions, solutions, capsules, patches, and the like. The most suitable formulation of a composition containing the compound of Formula (I) (e.g., a compound of Formula (Ia)) in any given case may depend on the severity of the condition being treated. The compositions may be conveniently presented in unit dosage form and prepared by any of the methods well known in the art of pharmacy. The compounds of Formula (I) (e.g., a compound of Formula (Ia)) may also be administered by controlled release means and/or delivery devices.

Pharmaceutical compositions comprising a formulation containing the compound of Formula (I) can be characterized by particles of a therapeutically effective size and shape. Particles containing the compound of Formula (I) for administration by inhalation desirably have an aerodynamic diameter permitting a patient to inhale and retain the particles at the therapeutically relevant site within the lung. For some indications, a particle size of greater than about 1 micron is desirable, to permit retention of the particle within the respiratory tract. A particle size of about 2-4 microns is suitable for treatment of certain indications (e.g., COPD), while a particle size of about 8 microns can be suitable for other indications (e.g., cough). In general, the particles containing the compound of Formula (I) for administration by inhalation desirably have an aerodynamic diameter of from 1-10 microns, preferably from about 2 to about 8 microns. If necessary, the size of particles obtained by crystallization may conveniently be reduced by micronization.

The term “aerodynamic particle size” is defined for the purposes of the present application as the diameter of a sphere of unit density which has the same settling velocity in air as the aerosol particle being measured (e.g., measured as an analytical parameter using a Cascade Impactor (CI)). Aerodynamic diameter is measured by a cascade impactor. The term “mass median aerodynamic diameter” or “MMAD” is defined as the median of the distribution of mass with respect to aerodynamic diameter. The median aerodynamic diameter and the geometric standard deviation are used to describe the particle size distribution of an aerosol, based on the mass and size of the particles. According to such a description, fifty percent of the particles by mass will be smaller than the median aerodynamic diameter, and fifty percent of the particles will be larger than the median aerodynamic diameter.

Pharmaceutical compositions comprising a formulation containing the compound of Formula (I) formulated for administration by inhalation can be delivered with a dry powder inhaler device. Dry powder inhalers (DPI's) are well known devices for administering pharmaceutically active agents to the respiratory tract. Consequently, they are particularly suitable when used for the administration of active agents in the treatment of diseases such as asthma, bronchitis, cough, chronic obstructive pulmonary disease (COPD), emphysema, rhinitis, etc. Since the drug acts directly on the target organ much smaller quantities of the active ingredient may be used, thereby minimizing any potential side effects. Dry powder compositions for use as inhalable medicaments in DPI's typically comprise a pharmaceutically active agent intimately admixed with an excess of pharmaceutically acceptable excipient or excipients (often called carrier(s)). Such excipients serve not only to dilute the quantity of active agent administered in each dose but also to establish acceptable manufacture of the powder mixture and aid in the aerosolisation of the drug. Such a high proportion of excipient will essentially determine the properties of the powder formulation, particularly the manufacturing characteristics.

Pharmaceutical preparations can be prepared in accordance with standard procedures selected to treat a condition that is mitigated, eliminated, prevented or otherwise treated by the administration of a compound to inhibit the TRPA1 ion channel (see, e.g., Remington's Pharmaceutical Sciences, Mack Publishing Company, Easton, Pa. and Goodman, and Gilman's “The Pharmaceutical Basis of Therapeutics,” Pergamon Press, New York, N.Y., the contents of which are incorporated herein by reference, for a general description of the methods for administering various therapeutic agents for human therapy). For example, the pharmaceutical compositions can be formulated for a desired route of administration, such as oral delivery. In particular, a medicament comprising a compound of Formula (I) (e.g., a compound of Formula (Ia)) can be formulated for oral administration for the therapeutic treatment of medical conditions, such as chronic or acute pain.

In preparing the compositions for oral dosage form, any of the usual pharmaceutical media may be employed as carriers, such as, for example, water, glycols, oils, alcohols, flavoring agents, preservatives, coloring agents, and the like in the case of oral liquid preparations (such as suspensions, solutions and elixirs) or aerosols; or carriers such as starches, sugars, micro-crystalline cellulose, diluents, granulating agents, lubricants, binders, disintegrating agents, and the like may be used in the case of oral solid preparations such as, for example, powders, capsules, and tablets, with the solid oral preparations being preferred over the liquid preparations. An example of a carrier is a cyclodextrin, for instance the sulfobutylether β-cyclodextrin compound available under the trade name Captisol® (CyDex Pharmaceuticals, Inc, Lenexa, Kans.). An example of a solid oral preparation is tablets or capsules containing the compound of Formula (I) (e.g., a compound of Formula (Ia)). If desired, tablets may be coated by standard aqueous or non-aqueous techniques.

The pharmaceutical compositions comprising one or more compounds of Formula (I) (e.g., a compound of Formula (Ia)) can be sterilized, for example, by filtration through a bacterial-retaining filter, or by incorporating sterilizing agents in the form of sterile solid compositions, which can be dissolved or dispersed in sterile water or other sterile injectable medium just prior to use.

A compound of Formula (I) described herein can be administered by inhalation. A pharmaceutical composition comprising a compound of Formula (I) can be provided in a metered dose aerosol dispenser containing an aerosol pharmaceutical composition for pulmonary or nasal delivery comprising an agent that inhibits a TRPA1-mediated current with an IC50 of 1 micromolar or less. For instance, it can be a metered dose inhaler, a dry powder inhaler or an air-jet nebulizer.

The pharmaceutical compositions comprising a compound of Formula (I) can also be admixed with solid or liquid pharmaceutically acceptable nontoxic carriers, diluents and adjuvants, including appropriate surfactants, in order to prepare the composition for use and to aid in administration to the patient by inhalation. Such pharmaceutical carriers can be sterile liquids, such as water and oils, including those of petroleum, animal, vegetable or synthetic origin. Water is a preferred carrier. Saline solutions can also be employed as liquid carriers. For example, a suitable dose amount of a compound of Formula (I) can be dissolved in saline solution at a desired concentration to form a pharmaceutical composition suitable for administration by the inhalation route. Surfactants such as polyoxyethylene fatty acid esters polyoxyethylene sorbitan acid esters, or glyceryl esters, for example, may be employed. Other suitable pharmaceutical carriers are described in Remington's Pharmaceutical Sciences by E. W. Martin. A pH in the range of about 4.5 to 5.5 is preferred. The pH can be adjusted with a conventional pharmaceutically acceptable buffer.

The inhaled pharmaceutical composition comprising a compound of formula (I) can be administered to the patient by means of a pharmaceutical delivery system for the inhalation route. The pharmaceutical delivery system is one that is suitable for respiratory therapy by topical administration of the imidazoline to mucosal linings of the tracheobronchial tree. For example, this invention can utilize a system that depends on the power of a compressed gas to expel the imidazoline from a container. See Sciarra et al, Theory and Practice of Industrial Pharmacy, 1976:270-295, which is relied upon and incorporated by reference herein. An aerosol or pressurized package can be employed for this purpose.

The pharmaceutical compositions containing a compound of Formula (I) can also be carried out with a nebulizer, which is an instrument that generates very fine liquid particles of substantially uniform size in a gas. Preferably, a liquid containing the imidazoline is dispersed as droplets about 5 mm or less in diameter in the form of a mist. The small droplets can be carried by a current of air or oxygen through an outlet tube of the nebulizer. The resulting mist penetrates into the respiratory tract of the patient.

A powder composition containing a compound of Formula (I), with or without a lubricant, carrier, or propellant, can be administered to a patient in need of therapy. This embodiment can be carried out with a conventional device for administering a powder pharmaceutical composition by inhalation.

### Administration of Compositions Comprising the Compound of Formula (I)

Pharmaceutical compositions containing the compound of Formula (I) (e.g., a compound of Formula (Ia)) or pharmaceutically acceptable salts thereof can be used to treat or ameliorate medical conditions responsive to the inhibition of the TRPA1 ion channel in subjects (e.g., humans and animals). For example, the pharmaceutical compositions comprising a compound of Formula (I) (e.g., a compound of Formula (Ia)), or a pharmaceutically acceptable salt thereof, are useful as a perioperative analgesic, for example in the management of mild to moderate acute post-operative pain and management of moderate to severe acute pain as an adjunct to opioid analgesics. For example, a compound of Formula (Ia) can be used in the manufacture of a medicament for the treatment of pain. Optionally, the medicament can also include or be indicated for use in combination with a second compound selected from the group consisting of opioids, non-steroidal anti-inflammatory agents, calcitonin gene-related peptide (CGRP)-antagonists and steroids.

The compounds of Formula (I) (e.g., a compound of Formula (Ia)) may also be used in combination with the administration of opioid analgesics. For example, the pharmaceutical compositions comprising a compound of Formula (I) (e.g., a compound of Formula (Ia)), or a pharmaceutically acceptable salt thereof, are useful as a perioperative analgesic given in combination with an opioid analgesic, for example in the management of mild to moderate acute post-operative pain and management of moderate to severe acute pain as an adjunct to opioid analgesics.

The pharmaceutical compositions comprising a therapeutically-effective dose of the compound of Formula (I) (e.g., a compound of Formula (Ia)) can be administered to a patient for treatment of pain in a clinically safe and effective manner, including one or more separate administrations of the pharmaceutical compositions comprising the compound of Formula (I) (e.g., a compound of Formula (Ia)). For example, a pharmaceutical composition, when administered to a subject, results in an alanine aminotransferase (ALT) and/or aspirate aminotransferase (AST) level of less than about 250 mg/dL (e.g., about 200 mg/dL, 150 mg/dL, 100 mg/dL or 50 mg/dL) three days after administration.

The amount of active ingredients which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about fifty percent of active ingredient. In one embodiment, this amount is 1.6% (weight to weight). In another embodiment, this amount is 40% (weight to volume). Pharmaceutical compositions can contain, for example, 1 to 50% of a compound of Formula (I) (e.g., a compound of Formula (Ia)) in combination with a pharmaceutically acceptable carrier.

Pharmaceutical compositions containing the compound of Formula (I) (e.g., a compound of Formula (Ia)) or pharmaceutically acceptable salts thereof can be used to treat or ameliorate pain. Methods of treating medical conditions responsive to the inhibition of the TRPA1 ion channel in subjects (e.g., humans and animals) can include the administration of a therapeutically effective amount of the compound of the Formula (I) (e.g., a compound of Formula (Ia)) or a pharmaceutically-acceptable salt thereof. The pain can be chronic or acute. Methods of treatment can include administering to a subject in need thereof a therapeutically-effective amount of the compound of Formula (I) (e.g., a compound of Formula (Ia)) or a pharmaceutically acceptable salt thereof in one or more doses over a course of treatment. The pharmaceutical compositions comprising a therapeutically-effective dose of the compound of Formula (I) (e.g., a compound of Formula (Ia)) can be administered to a patient for treatment of pain in a clinically safe and effective manner, including one or more separate administrations of the pharmaceutical compositions comprising one or more compounds of Formula (I) (e.g., a compound of Formula (Ia)). For example, a pharmaceutical composition, when administered to a subject, results in an ALT and/or AST level of less than about 250 mg/dL (e.g., about 200 mg/dL, 150 mg/dL, 100 mg/dL or 50 mg/dL) three days after administration.

According to a further aspect, the invention provides the compound of Formula (I) (e.g., a compound of Formula (Ia)), or a pharmaceutically acceptable salt thereof, for the treatment or amelioration of pain or providing analgesia.

According to a further aspect, the invention provides the compound of Formula (I), or a pharmaceutically acceptable salt thereof, as a medicament.

In one example, the compound of Formula (I) (e.g., a compound of Formula (Ia)) can be orally administered to a subject human. The total daily dose of a compound of Formula (I) (e.g., a compound of Formula (Ia)) can be about 0.1 mg/kg/day to about 100 mg/kg/day of the compound of Formula (I) (e.g., a compound of Formula (Ia)) administered orally to a subject one to four times a day (e.g., QD, BID, TID, or QID) (e.g., 0.1 mg/kg/day to about 50 mg/kg/day). The total daily dose administered to a human can also be about 1 mg/kg/day to about 25 mg/kg/day, or about 3 mg/kg/day to about 10 mg/kg/day. The amount per administered dose or the total amount administered will depend on such factors as the nature and severity of the pain, the age and general health of the patient, and the tolerance of the patient to the compound.

A drug product comprising the compound of Formula (I) (e.g., a compound of Formula (Ia)) can be prepared by a suitable formulation process, e.g., wet granulation (see Remmington pharmaceutical sciences). The pharmaceutical composition can be a unit dose in a shape to facilitate swallowing (e.g., a 0 or 00 size capsule). The unit dose can have an amount of the pharmaceutical composition ranging from 100 to 1600 mg in a size “00” capsule (e.g., from 100 to 800 mg) or equivalent tablet size. If 500 mg active/unit dose is achieved then development for that technology will be targeted to the highest achievable dose. Individual unit dosage forms can include, for example, 200 mg, 400 mg, 800 mg, 1200 mg or 1600 mg of a compound of Formula (I) formulated for oral administration.

For example, a pharmaceutical composition comprising a therapeutically effective dose of the compound of Formula (I) (e.g., a compound of Formula (Ia)) or a pharmaceutically acceptable salt thereof can be administered (e.g., orally) to a subject in need thereof multiple times per day (e.g., BID) over a course of treatment of one or more days to treat pain in the subject.

Pharmaceutical compositions comprising a compound of Formula (I) (e.g., a compound of Formula (Ia), a compound of Formula (Ib) and/or a combination of compounds of Formula (Ia) and (Ib)) are useful for administration for the treatment of respiratory conditions, such as obstructive diseases, e.g., chronic obstructive pulmonary disease (COPD), asthma (e.g., cold induced asthma, exercise-induced asthma, allergy-induced asthma, and occupational asthma), and cough.

A method for treating or ameliorating asthma in an animal or human is provided, comprising administering to the animal or human a pharmaceutical composition comprising a therapeutically effective amount of a compound of Formula (Ia), or a pharmaceutically acceptable salt thereof, by inhalation. In one example of this method, the compound of Formula (Ia) is in the form of a hydrochloride salt. The asthma can be allergic asthma. The pharmaceutical composition can be administered as an aerosol. The pharmaceutical composition can be administered using a medical nebulizer. The compound of Formula (Ia) can be administered in the form of a hydrochloride salt. The pharmaceutical composition can be administered at a dose of about 0.5-25 mg/kg.

### EXAMPLES

Certain examples below illustrate the synthesis of the compound of Formula (I) (e.g., a compound of Formula (Ia)) and a pharmaceutically acceptable salt thereof. Further, the disclosure includes variations of the methods described herein to produce the compounds of Formula (I) (e.g., a compound of Formula (Ia)) that would be understood by one skilled in the art based on the instant disclosure.

### Example 1A

### Synthesis of the Compound of Formula (Ia)

### Step 1

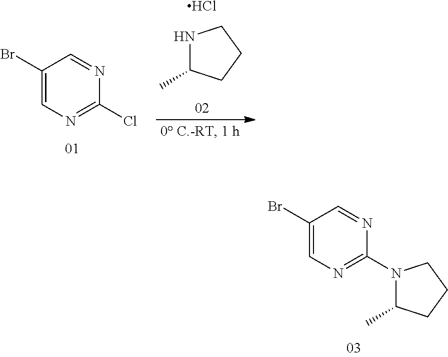

A dry 1 L round bottom flask charged with (S)-2-methylpyrrolidine (compound 02) (44.2 mL, 465 mmol) was cooled to 0° C. Compound 01 (60 g, 310 mmol) was added to the cooled amine compound 02 over 2 minutes (observed extreme exotherm). After addition was complete, the reactants were warmed to room temperature and continued to stir for 1 hr. Followed by liquid chromatography mass spectrometry (LCMS) and ultra-performance liquid chromatography (UPLC).

The resulting orange solids were dissolved in (9:1 DCM:MeOH, 200 mL), washed with saturated sodium bicarbonate 150 mL and water (3×100 mL). The combined aqueous layers were back extracted with (9:1 DCM:MeOH). The combined organic layers were washed with brine, dried over MgSO₄, and concentrated onto silica. The column was purified using a 400 g silica column with (Hex: EtOAc) solvent system (0% 4CV; 0-30% 6CV; 30% 6CV). The product eluted between 20-30% EtOAc. Fractions containing product were combined and dried under vacuum, the resulting clear oil was treated with hexanes, agitated, and then evaporated. A fine crystal formation was observed. The fine crystal formation was allowed to stand at 0° C. to aide white crystalline solids of compound 03.

For compound 03 in Step 1, Example 1: Isolated Yield: 67.2 g (89%) as white crystalline solids. (m/z M+=241); ¹H NMR (300 MHz, DMSO) δ 9.01 (s, 1H), 8.42 (s, 2H), 4.20-4.06 (m, 1H), 3.56-3.34 (m, 2H), 2.12-1.81 (m, 3H), 1.68 (s, 1H), 1.16 (d, J=6.3 Hz, 3H).

### Step 2

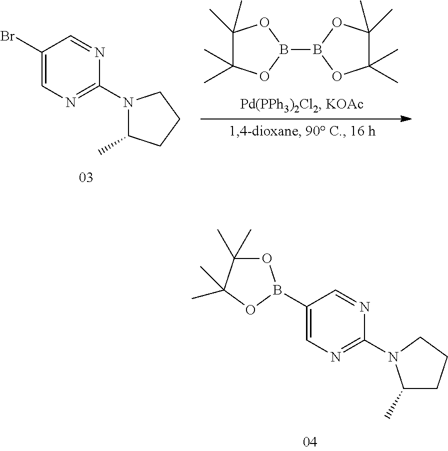

A 2 L three neck round bottom flask was charged with compound 03 (45 g, 186 mmol), bis(pinacolato)diboron (65.2 g, 257 mmol), bis(triphenylphosphine)palladium chloride (13.05 g, 18.59 mmol), potassium acetate (36.5 g, 372 mmol) and suspended in anhydrous 1,4-dioxane (Volume: 929 mL). The flask was flushed with nitrogen and the solids were fitted with reflux condenser and heated to 90° C. overnight.

1,4-dioxane was removed in vacuo. The crude material was dissolved in DCM (200 mL) and washed with water (3×100 mL). Combined aqueous layers back extracted with EtOAc. The combined organic layers were washed with brine, dried over MgSO₄, and concentrated onto silica. Material was split into two batches and column purified using 200 g silica column with Hex:EtOAc solvent system (0% CV; 3% 8CV; 5-20% 10CV; 20-50% 5CV). The starting material eluted with 3% EtOAc while desired product eluted between 5-40% EtOAc. Fractions containing product were combined and solvent was removed in vacuo to afford compound 04.

For compound 04 in Step 2, Example 1: Isolated Yield: 23.0 g (42%) as off-white solids. [(m/z=M+=289.20 (boronic acid observed at m/z 207.12)); ¹H NMR (300 MHz, DMSO) δ 8.45 (s, 2H), 4.31-4.17 (m, 1H), 3.62-3.38 (m, 2H), 2.12-1.81 (m, 3H), 1.73-1.61 (m, 1H), 1.27 (s, 12H), 1.17 (d, J=6.3 Hz, 3H).

### Step 3

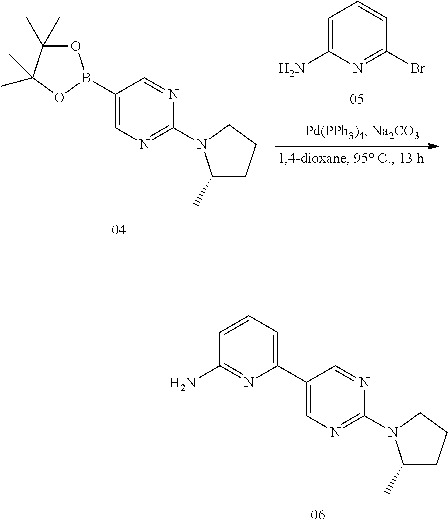 [SMILES: C[C@H]1CCCN1c1ncc(B2OC(C)(C)C(C)(C)O2)cn1 ; Nc1cccc(Br)n1 ; C[C@H]1CCCN1c1ncc(-c2cccc(N)n2)cn1]

A 1L round bottom flask was charged with compound 05 (15.14 g, 87 mmol), compound 04 (23.00 g, 80 mmol), purged with nitrogen, and followed by an addition of Pd(PPh₃)₄ (9.19 g, 7.95 mmol). The solids were suspended in a mixture of anhydrous 1,4-dioxane (398 ml) and aqueous 2M sodium carbonate (119 mL, 239 mmol). Reaction was heated to 95° C. for 13 hours.

Organics were separated from salts by transfer of liquid phase to 2 L round bottom flask. Salts were rinsed with 1,4-dioxane and combined with previously separated 1,4-dioxane solution. 1,4-dioxane was removed under vacuo. The yellow crude residue was dissolved in DCM and washed with water (3×100 mL), brine, and dried over MgSO₄ then concentrated onto silica. The column was purified using a 200 g silica column with DCM:EtOAc solvent system (0% 20CV; 20% 10 CV; 50-80% 10CV; 80% 5CV). The desired product eluted between 50-80% EtOAc. The fractions containing product were concentrated to isolate the compound 06.

For compound 06 in Step 3, Example 1: Isolated Yield: 13.7 g (67%) as light yellow solids. (m/z=M+=255.15); ¹H NMR (300 MHz, DMSO) δ 8.88 (s, 2H), 7.40 (t, J=7.8 Hz, 1H), 6.95 (d, J=7.1 Hz, 1H), 6.35 (d, J=7.9 Hz, 1H), 5.96 (s, 2H), 4.31-4.19 (m, 1H), 3.66-3.41 (m, 2H), 2.13-1.84 (m, 3H), 1.75-1.65 (m, 1H), 1.22 (d, J=6.3 Hz, 3H).

### Step 4

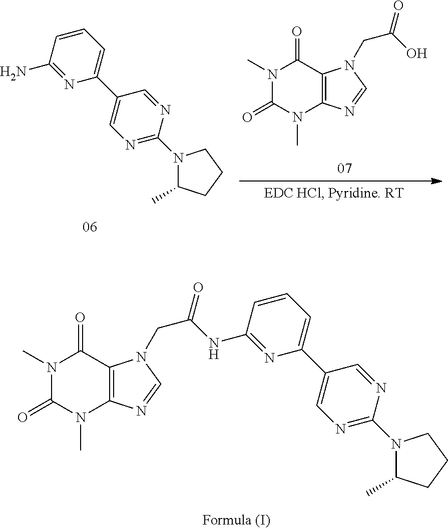 [SMILES: C[C@H]1CCCN1c1ncc(-c2cccc(N)n2)cn1 ; Cn1c(=O)c2c(ncn2CC(=O)O)n(C)c1=O ; C[C@H]1CCCN1c1ncc(-c2cccc(NC(=O)Cn3cnc4c3c(=O)n(C)c(=O)n4C)n2)cn1]

A dry 200 mL round bottom flask was charged with compound 07 (12.17 g, 51.1 mmol), compound 06 (13.7 g, 53.7 mmol), EDC (19.59 g, 102 mmol) flushed with nitrogen followed by the addition of anhydrous pyridine (128 ml) (no exotherm observed). The suspension was stirred at room temperature for 1 h.

The reaction mixture was diluted with 100 mL water. An off-white precipitation was observed. The suspension was transferred to a 500 mL flask charged with stir bar and diluted with 150 mL 0.1M HCl while stirring. The precipitate turned light red in color forming an amorphous solid. Aqueous formulation was extracted with EtOAc (3×100 mL). The organic layer was washed with 0.1M HCl (3×50 mL), water, brine, and dried over MgSO₄ then concentrated onto silica. The column was purified using DCM:MeOH solvent system (0% 5CV; 0-3% 10CV; 3-4% 4CV; 4% 10CV). The product eluted between 3-4% MeOH. Appropriate fractions were pooled, and solvents were removed in vacuo, and was placed on high vacuum to afford the compound of Formula (Ia).

For the compound of Formula (Ia) in Step 4, Example 1: Isolated Yield: 20.7 g (85%) as off-white solids. The compound Formula (I) (m/z=M+=475), ¹H NMR (300 MHz, DMSO) δ 10.95 (s, 1H), 9.01 (s, 2H), 8.09 (s, 1H), 7.82 (t, J=7.6 Hz, 2H), 7.61 (d, J=8.4 Hz, 1H), 5.32 (s, 2H), 4.33-4.23 (m, 1H), 3.71-3.49 (m, 2H), 3.47 (s, 3H), 3.20 (s, 2H), 2.18-1.84 (m, 3H), 1.70 (m, 1H), 1.24 (d, J=6.3 Hz, 3H).

### Example 1B

### Synthesis of Deuterated Compound of Formula (Ia)

A deuterated compound (12) was prepared as described in FIG. 1B. Compound 10 was prepared from a commercial starting material compound 08 according to the following procedure:

Theophiline-d6 (0.480 g, 2.58 mmol) and potassium carbonate (0.392 g, 2.84 mmol), were suspended in DMF (12.89 mL), followed by addition of ethyl 2-chloroacetate (0.275 mL, 2.58 mmol) and heated to 90° C. for 1 hr. The reaction mixture was cooled to room temperature and diluted into 15 mL stirred water solution at room temperature. To the aqueous solution, lithium hydroxide (0.123 g, 5.16 mmol) in 10 mL water was added and continued to stir at room temperature for 1 hr. The solution was titrated to pH 4 with 5M HCl aq. The resulting white solids were collected via vacuum filtration to afford compound 10 (0.510 g, 81%) ESI-MS (EI+, m/z): 244.11

Deuterated compound 12 was synthesized in the same manner as Formula (Ia) using compound 06 (0.150 g, 0.609 mmol), and compound 10 (0.163 g, 0.640 mmol). The resulting crude solids were collected via vacuum filtration. Column purified by silica gel chromatography to afford deuterated compound 12 (0.135 g, 46%) ESI-MS (EI+, m/z): 481.25. ¹H NMR (300 MHz, DMSO) δ 10.95 (s, 1H), 9.01 (s, 2H), 8.08 (s, 1H), 7.82 (t, J=7.7 Hz, 2H), 7.61 (d, J=8.5 Hz, 1H), 5.76 (s, 1H), 5.32 (s, 2H), 4.29 (s, 1H), 3.69-3.56 (m, 1H), 3.53 (s, 1H), 2.13-1.85 (m, 3H), 1.71 (d, J=2.3 Hz, 1H), 1.24 (d, J=6.3 Hz, 3H).

In addition to compound 12, the compounds described herein also include isotopes of the compound of Formula (I) (e.g., a compound of Formula (Ia)). For example, isotopes of Formula (I) (e.g., a compound of Formula (Ia)) can be formed as molecules formed by substitution of atomic isotopes at one or more of the atoms that constitute the compound of Formula (I) (e.g., a compound of Formula (Ia)). For example, the isotopes of Formula (I) may be radiolabeled with radioactive isotopes. Isotopes of Formula (I) (e.g., a compound of Formula (Ia)) include compounds formed by substitution of hydrogen in Formula (I) (e.g., a compound of Formula (Ia)) with deuterium (²H), or tritium (³H), or substitution of one or more carbon atoms in Formula (I) (e.g., a compound of Formula (Ia)) with carbon-13 (¹³C) or carbon-14 (¹⁴C). Preferred isotopes of Formula (I) (e.g., a compound of Formula (Ia)) inhibit TRPA1 in humans or animals. All isotopic variations of the compounds disclosed herein, whether radioactive or not, are intended to be encompassed within the scope of the present invention. For example, deuterated compounds or compounds containing ¹³C are intended to be encompassed within the scope of the invention.

### Example 1C

### Synthesis of the Compound of Formula (Ib) (the Enantiomer of Formula (Ia))

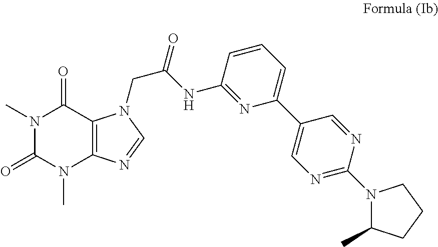 [SMILES: C[C@@H]1CCCN1c1ncc(-c2cccc(NC(=O)Cn3cnc4c3c(=O)n(C)c(=O)n4C)n2)cn1]

The compound of Formula (Ib) (enantiomer of Formula Ia) was synthesized using an identical procedure as described above, with the one difference being the use of (R)-2-methylpyrrolidine as a starting material in step 1 instead of (S)-2-methylpyrrolidine (Compound 02 in FIG. 1A). The yield in the last step is 92%, as white powder. ¹H NMR (300 MHz, DMSO-d6) δ 10.95 (s, 1H), 9.01 (s, 2H), 8.09 (s, 1H), 7.82 (t, J=7.7 Hz, 2H), 7.61 (d, J=8.5 Hz, 1H), 5.32 (s, 2H), 4.47-4.13 (m, 1H), 3.72-3.58 (m, 2H), 3.27 (s, 3H), 3.20 (s, 3H), 2.17-1.86 (m, 3H), 1.71 (s, 1H), 1.24 (d, J=6.3 Hz, 3H). LCMS (m/z=M+H=476).

The IC50 for hTrpA1 measured in 1% RSA was 15.2 μM for formula (Ib), compared to 5.3 μM for the compound of Formula (Ia).

### Example 2

### Formation of the HCl Salt of a Compound of Formula (Ia)

1M HCl in EtOH: A 500 mL flask was charged with stir bar, and 185 mL 200 proof EtOH at 0° C. Acetyl chloride (14.20 mL, 200 mmol) was then added and stirred at 0° C. for five minutes, then at room temperature for 10 minutes.

HCl Salt Precipitation: To a 1L round bottom flask was charged with the dry compound of Formula (I) (20.5 g, 43.1 mmol) and 200 mL 1M HCl in EtOH (freshly made) was added and stirred at room temperature for 1 hr. The suspension went from a mostly homogenous clear yellow to white solid suspension in light yellow solvent. After 1 hr, solids were collected via vacuum filtration with aide of EtOH, then rinsed with EtOH (3×100 mL) and placed on high vacuum overnight. After 18 hours, material was removed from high vacuum and transferred to amber jar.

For Example 2, HCl Salt Isolated Yield: 22.6 g (>100%) as an off-white solid. (I) salt (m/z=M+=475), ¹H NMR (300 MHz, DMSO) δ 10.95 (s, 1H), 9.01 (s, 2H), 8.09 (s, 1H), 7.82 (t, J=7.6 Hz, 2H), 7.61 (d, J=8.4 Hz, 1H), 5.32 (s, 2H), 4.33-4.23 (m, 1H), 3.71-3.49 (m, 2H), 3.47 (s, 3H), 3.20 (s, 2H), 2.18-1.84 (m, 3H), 1.70 (m, 1H), 1.24 (d, J=6.3 Hz, 3H). Elemental analysis: C, 50.54 (cal. 53.96); H, 5.34 (cal. 5.12); Cl, 6.34 (cal. 6.92); N, 22.69 (cal. 24.62); O, 9.38.

### Example 3

### Measuring in vitro Inhibition of TRPA1

The in vitro inhibition of TRPA1 of the compound of Formula (Ia) was tested using the procedure outlined in del Camino et al., J. Neurosci., 30(45):15165-15174, incorporated herein by reference and described below. Data for TRPA1 inhibition and the selectivity of TRPA1 inhibition was obtained by this method for the compound of Formula (Ia) and included in Table 1 and Table 2. All currents were recorded in whole-cell configuration using EPC-9 and EPC-10 amplifiers and Patchmaster software (HEKA). Patch pipettes had a resistance of 1.5-3 MΩ and 60-75% of the series resistance was compensated. The standard pipette solution consisted of 140 mM CsAsp, 10 mM EGTA, 10 mM HEPES, 2.27 mM MgCl₂, 1.91 mM CaCl₂, 4 mM MgATP, and 0.1-0.3 mM Na₂GTP, with pH adjusted to 7.2 with CsOH. In addition, a solution containing 145 mM CsCl, 10 mM HEPES, 10 mM EGTA and 1 mM MgCl₂ (pH 7.2 adjusted with CsOH) can be used. The standard bath solution contained 150 mM NaCl, 10 mM HEPES, 10 mM glucose, 4.5 mM KCl, 1 mM EGTA, 3 mM MgCl₂, with pH adjusted to 7.4 with NaOH. In some instances, 2 mM CaCl₂ was added in place of EGTA and the concentration of MgCl₂ was reduced to 1 mM.

Data were collected either by continuous recordings at −60 mV or by applying voltage ramps from a holding potential of 0 mV every 4 s. Continuous recordings were collected at 400 Hz and digitally filtered off-line at 10 Hz for presentation. Voltage ramps were applied from −100 mV to 100 mV over the course of 400 ms, and data were collected at 10 kHz and filtered at 2.9 kHz. Inward and outward currents were analyzed from the ramps at −80 and 80 mV, respectively. Liquid junction potential correction was not used.

Solutions were switched using a gravity-fed continuous focal perfusion system. To achieve rapid temperature changes, two temperature control and perfusion systems were employed simultaneously. For temperatures ≧22° C., a Warner Instruments bipolar temperature controller (TC-344B) and inline heater (SHM-8) were used. For temperatures below 22° C. a Warner Instruments temperature controller (CL-100) and thermal cooling module (TCM-1) were used. Temperatures were confirmed using a thermistor (Warner Instruments, TA-29), with temperatures at the recorded cell estimated to be within +/−2° C. of those reported.

IC₅₀ of compounds was estimated by testing each compound at 5 micromolar and 500 nanomolar. When 5 micromolar compound showed no block, IC₅₀ was estimated as >10 micromolar. When 5 micromolar compound showed 50% or less block, a rough estimate of IC₅₀ in the range of 5-10 micromolar could be made. IC₅₀ for compounds between 500 nanomolar and 5 micromolar was similarly estimated. Compounds blocking 50% or more at 500 nanomolar are retested at multiple concentrations, and the % block at each is fitted by standard equations to determine IC₅₀ accurately using a 5-6 point concentration/response experiment.

### Example 4

### Evaluating the In Vivo Efficacy of TRPA1 Inhibitor Compounds

The compound of Formula (Ia) was evaluated for activity in vivo. In some examples, comparator TRPA1 inhibitor compounds of Formula (II) or Formula (III) were also evaluated, as described in the examples below.

The comparator compound of Formula (II) and methods of making and using this compound are disclosed as the TRPA1 inhibitor compound 200 in U.S. Pat. No. 7,671,061 (filed Dec. 22, 2006, issued Mar. 2, 2010) and are incorporated herein by reference in their entirety.

The comparator compound of Formula (III) and methods of making and using this compound are disclosed as the TRPA1 inhibitor compound of Formula (I) in PCT patent application PCT/US2009/069146 (published as WO2010/075353A1 on Jul. 1, 2010) and are incorporated herein by reference in their entirety.

The potency and pharmacokinetic (PK) properties of (a) the compound of Formula (Ia); and (b) comparator compound of Formula (III) were evaluated. Bioavailability was measured as well. A pharmacokinetic study was performed to obtain a plasma drug concentration vs time plot for the drug after both intravenous (IV) and oral (PO) administration. The absolute bioavailability is the dose-corrected area under curve (AUC) non-intravenous divided by AUC intravenous. The formula for calculating F for a drug administered by the oral route (PO) is given below.

The bioavailability was calculated using the equation shown below:

% F=AUC PO×Dose IV/AUC IV×Dose PO

### Human Plasma Protein Binding

The amount of compound in buffer (free fraction) and the amount of compound associated with the plasma fraction is determined by equilibrium dialysis; the amount of compound bound is expressed as a percentage. (Banker et al., Journal of Pharmaceutical Sciences (2003) 92(5): 967-74.)

In Table 6, an “A” indicates an IC₅₀ of less than 25 nanomolar; a “B” indicates an IC₅₀ of 25 nanomolar to less than 50 nanomolar; a “C” indicates an IC₅₀ of 50 nanomolar to less than 100 nanomolar; a “D” indicates an IC₅₀ of 100 nanomolar or greater.

While the compound of Formula (III) was more potent in vitro, the compound of Formula (Ia) has in vivo properties that make it advantageous over the compound of Formula (III). Greater protein binding was observed for the Compound of Formula (III) than the compound of Formula (Ia). IC₅₀ for the compound of Formula (Ia), when tested against hTRPA1, was between 50 and 100 nanomolar. The compound of Formula (Ia) was less than 99% protein-bound and the bioavailability for fed rats was greater than 50%. Although the IC₅₀ for the compound of Formula (III), when tested against hTRPA1, was between 0 and 25 nanomolar. The compound of Formula (III) was greater than 99% protein-bound and the bioavailability for fed rats was between 1 and 25%.

TABLE 6

| Parameter | Formula (III) | Formula (Ia) |
|---|---|---|
| Potency (IC₅₀) | | |
| Human | A | C |
| Rat | C | D |
| Dog | A | D |
| Bioavailability (Rat) | | |
| Fed | Between 1 and 25% | Greater than 50% |
| Fasted | Between 25 and 50% | Between 25 and 50% |
| Human Plasma Protein | Greater than 99% | Less than 99% |
| Binding | | |

In addition, as shown in Table 6 above, the compound of Formula (Ia) demonstrates less of a fed/fasted effect than the compound of Formula (III). Compounds with reduced fed/fasted effects in humans can lead to increased patient compliance. In addition, the compound of Formula (Ia) is less protein-bound than the compound of Formula (III). As a consequence, more of the compound is available to be distributed to the target tissues upon administration.

### Example 5

### Formalin-Induced Pain Behavior In Vivo Rodent Model

The compound of Formula (Ia) and the comparator compounds of Formula (II) and Formula (III) were tested in the formalin-induced pain test reported by Dubuisson et al., Pain 1977 December; 4(2):161-74 (incorporated herein by reference in its entirety). Dubuisson et al. (1977) describe a method for assessing pain and analgesia in rats and cats. Briefly, dilute formalin (50 μL of 3% formalin) is injected into the plantar surface of the hind paw. The animal is promptly returned to an observation arena (standard Plexiglass rat cage), at which point a trained observer records the time the animal spends exhibiting pain behaviors (flinching, licking, biting of the injected paw/leg) for a period of 5 minutes. The individual responsible for counting the pain behaviors in a particular study is blinded to the treatment groups.

Rats were treated with the HCl salt of Compound (Ia) at various doses (3, 10, 30, and 50 mg/kg, IP) or with the vehicle (IP). The vehicle animals showed an average of about 88.6 seconds exhibiting pain behaviors (e.g., flinching, lifting and licking the paw). Results are shown in FIG. 2 and Table 3a. The animals treated with Formula (Ia) showed a range of 5.9 to 85.8 seconds exhibiting pain behaviors. Results are shown in FIG. 2 and Table 3a. Results of Formula (II) effects on formalin induced responses are shown in Tables 3a and 3c. The animals treated with Formula (III) exhibited pain behaviors for 44.3 seconds compared to vehicle at 77.2 seconds. Results are shown in Table 3b.

### Example 6

### Complete Freund's Adjuvant (CFA) Inflammatory In Vivo Rodent Pain Model

The compound of Formula (Ia), the comparator compound of Formula (II) and ketoprofen were tested by the CFA-induced pain test method reported in del Camino et al., J. Neurosci., 30(45):15165-15174, incorporated herein by reference in its entirety.

Briefly, the hind paw was sensitized to cold temperature (allodynic), by administering 0.1 mL of Complete Freund's Adjuvant (CFA) to the left hind paw. 2-3 days later, the time taken for the animal to lift its CFA-injected paw was recorded compared to its un-injected normal right hind paw. Animals were placed on the surface of the cold plate (1° C.) and the operator stopped testing at the instant when the animal displays discomfort by flinching or lifting its paw from the plate (paw withdrawal latency, or PWL). To avoid tissue damage the maximum cut-off time was 5 minutes. Animals that were allodynic (average PWL to the first three pain behaviors <150 seconds for the CFA-injected hind paw: ˜≧50% difference between the normal and CFA-injected paw) were included in the study and subsequently randomized across treatment groups. The following day, the animals were dosed under blinded conditions. Following the 1-2 hour pre-treatment time, the post-dose PWL readings were again taken. The efficacy of the drug treatment was assessed by comparing the PWL in the drug treatment animals to those animals that received the vehicle.

### Example 7

### Surgical Incision Pain Behavior in vivo Rodent Model (FIG. 4)

The compound of Formula (Ia), the comparator compound of Formula (III) and ketoprofen were tested by the incisional pain test method reported in Brennan et al., Pain, 1996 March; 64(3):493-501 incorporated herein by reference in its entirety. Briefly, in rats under anesthesia, a 1 cm incision through skin and underlying muscle was made in the bottom of one hind paw. The incision was sutured closed and the animals allowed to regain consciousness in their home cage before being placed on a special mesh rack. The blinded observer subjectively assessed and recorded each animal's pain score every 5 minutes for 1 hour. Pain scores were assigned as follows: Score of 0=Injured paw was held flat on the rack and was bearing weight (=uninjured paw); 1=Injured paw was slightly lifted from the rack but was bearing some weight; 2=Injured paw was flat but was bearing no weight, or heel was lifted high off the rack with only toes touching. At the end of each hour, pain scores were added up and the final score recorded (maximum score=39). In a typical study the efficacy of the drug treatment was determined by comparing the cumulative guarding scores at 1-2 and 3-4 hours following surgical injury to the cumulative guarding scores of animals that received the vehicle.

Sixty (60) mg/kg delivered intraperitoneally (2 doses of 30 mg/kg before and immediately after the surgery) reduced spontaneous pain for up to 4 hours after surgery, equivalent to ketoprofen (2 doses of 2 mg/kg intraperitoneally). Thirty (30) mg/kg compound of Formula (Ia) intraperitoneally (2 doses of 15 mg/kg before and immediately after the surgery) only reduced spontaneous pain for up to 2 hours after surgery.

### Example 8

### Hepatotoxicity Serum Biomarker Study of the Compound of Formula (I) and a Comparator Compound of Formula (III)

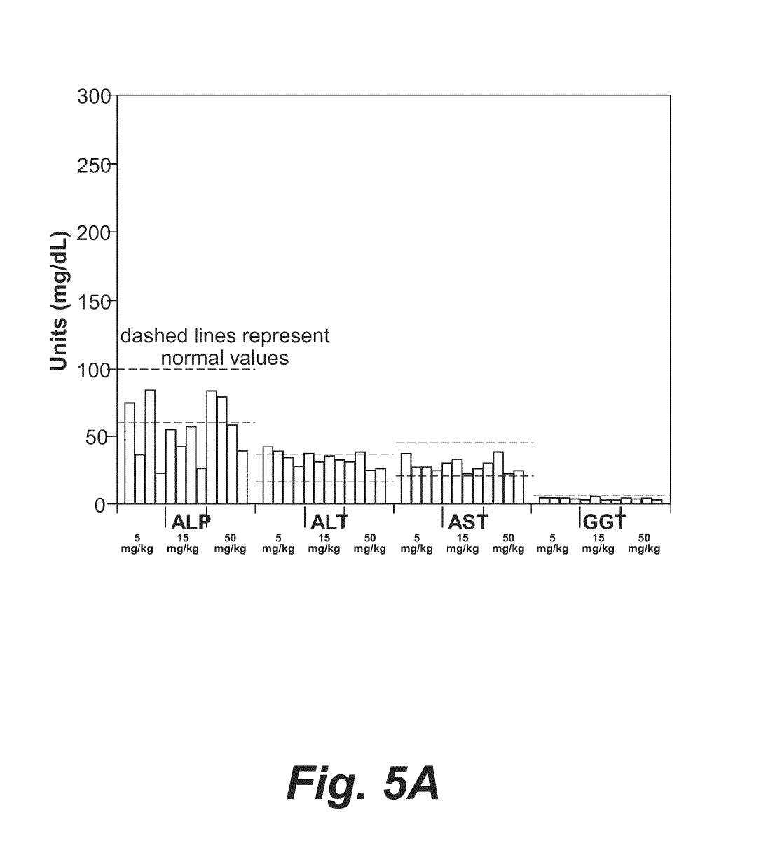

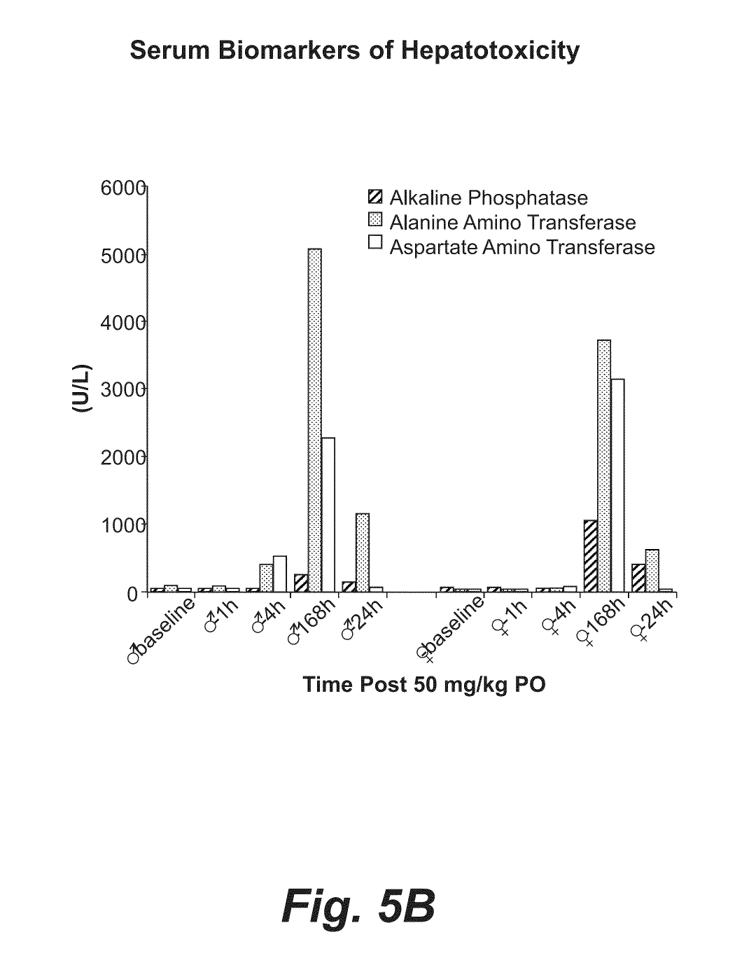

The compound of Formula (Ia) was orally dosed to female dogs at dose levels of 5, 15 or 50 mg/kg using 30% Sulfobutylether β-cyclodextrin as the vehicle for assessment of safety as measured via serum chemistry biomarkers of hepatotoxicity or bile duct injury FIG. 5A, showing measurements of alanine aminotranferease [ALT], aspartate aminotranferease [AST], alkaline phosphatase [ALP] and gamma-glutamyl transferase [GGT] in the dogs at each dose level (each bar represents a measurement from 1 dog in the study). The data in FIG. 5A shows that the compound of Formula (Ia) did not elevate serum biomarkers of hepatotoxicity or acute phase response when dosed at 50 mg/kg PO (oral).

In contrast, the data in FIG. 5B shows that the comparator compound of Formula (III) did elevate serum biomarkers of hepatotoxicity. For example, the ALT levels were elevated up to about 60-fold in male dogs and up to about 130-fold in female beagle dogs following a single PO dose of 50 mg/kg.

### Example 9

### Rodent Repeat Dose Toxicity Studies, Intraperitoneal (i.p.)

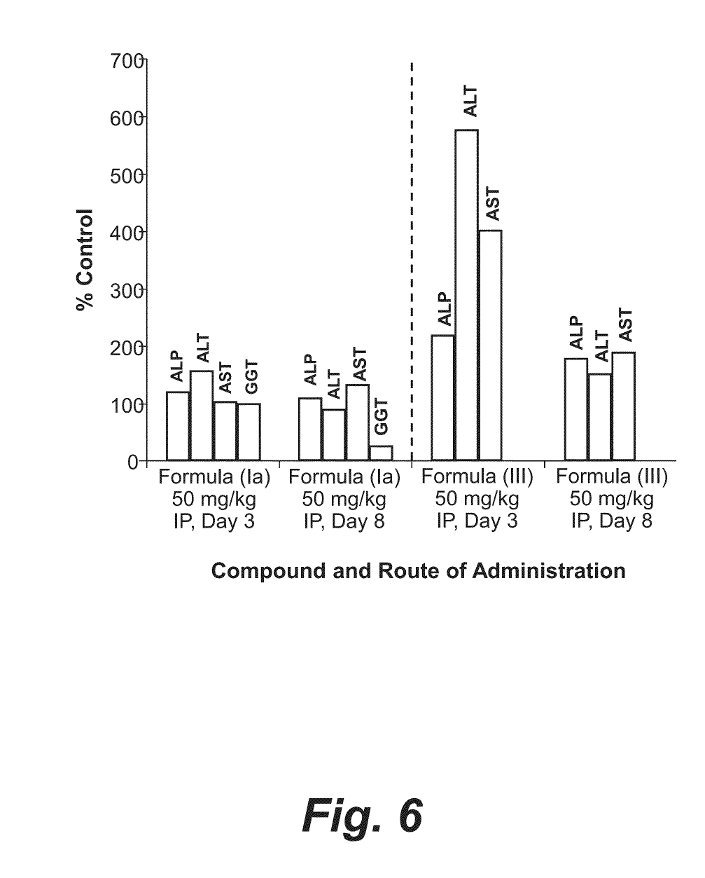

The compound of Formula (Ia) was evaluated in a 7-day repeat dose screening toxicity study in female rats. In order to maximize systemic exposure, rats were administered compound of Formula (Ia) i.p. at 50 mg/kg/day for 7 consecutive days, to obtain the results shown in FIG. 6. Clinical chemistry parameters were evaluated on Days 3 and 8. Histopathology was performed on select organs including the liver, kidney, spleen, and lung. After administration of the compound of Formula (Ia) at the 50 mg/kg IP dose, no adverse clinical signs, changes in body weight, or changes in clinical chemistry parameters were noted. No histopathological findings in the liver, kidney, spleen, or lung were observed after administration of the compound of Formula (Ia).

According to the pathologist's report, no adverse effects related to the compound of Formula (Ia) were identified in sections of liver harvested on study days 3 and 8 or spleen, kidney and lung harvested on study day 8.

In contrast, the data in FIG. 6 for compounds of Formula (III) shows that the comparator compound of Formula (III) did elevate serum biomarkers of hepatotoxicity as compared to Formula (Ia) following the 7-day repeat dose of 50 mg/kg/day for 7 consecutive days.

### Example 10

### Pharmaceutical Composition Containing the Compound of Formula (Ia)

The components of a pharmaceutically acceptable formulation can include a compound of Formula (Ia) as the active ingredient, hydroxypropyl-β-cyclodextrin (HPBCD) as a solubilization and stabilization agent and HCl as the pH adjustor. The formulated dosing solution can comprise 10 mg/mL of the compound of Formula (Ia) and 25% (w/v) HPBCD dissolved in 0.1N hydrochloric acid (HCl), pH 2.0. The formulation can be converted to a lyophilized dosage form for reconstitution prior to dosing at the clinical site.

A drug product comprising the compound of Formula (Ia) can be a prepared by dissolving the compound of Formula (Ia) as the drug substance (DS) in 25% w/v HPBCD in a 0.1M HCl solution at a final target pH of 2 (±0.5). The compounded solution can be filled into vials for subsequent lyophilization.

Optionally, the pharmaceutical compositions comprising a compound of Formula (I) can be formed as nanosuspensions, co-crystals, spray dried dispersions and hot melt extrusions. These technologies can be selected based on their utilization and demonstrated success for BCS class II drug compounds. The feasibility assessment of the selected drug delivery technologies can be conducted using the HCl salt form of the compound of Formula (Ia) in Example 2.

The pharmaceutical composition can be a unit dose ranging from 200 to 500 mg in a size “00” capsule or equivalent tablet size. If 500 mg active/unit dose is achieved then development for that technology will be targeted to the highest achievable dose.

Preferably, the pharmaceutical compositions comprising the compound of Formula (Ia) can be formulated to provide a reduction in pain following surgery (e.g., management of pain following surgery compared to placebo to achieve about 50-100% reduction in opiate use within the first 24 hours after surgery). The pharmaceutical compositions comprising the compound of Formula (Ia) can be indicated for use for treatment of pain, including use as an orally administered analgesic and/or in compositions formulated for the treatment of pain caused by inflammation (e.g., to block acute pain and prevent or reduce inflammation at a wound site and prevent central sensitization). In one embodiment, the pharmaceutical compositions comprising the compound of Formula (Ia) can be administered BID for a suitable time period (e.g., 7-14 days) and provide analgesia within about 30 minutes of administration. Preferably, the pharmaceutical composition(s) comprising the compound of Formula (Ia) can provide clinically measurable decreases in pain scores, without respiratory depression and/or drug-induced CNS effects.

### Example 11

### Single Ascending Dose Phase 1A Study

A randomized, double-blind, placebo-controlled, cross-over, single dose, safety, tolerability and pharmacokinetic study of 6 ascending doses of the compound of Formula (Ia) was undertaken in two cohorts of healthy male volunteers. A total of eighteen eligible healthy mail volunteers were recruited utilizing an alternating panel design. The first cohort of nine subjects (Cohort 1) was sequentially enrolled into 3 of 6 dosing periods (Dose Levels 1, 3, and 5). The remaining cohort of nine subjects (Cohort 2) was enrolled in the other 3 dosing periods (Dose Levels 2, 4, and 6). Within each dosing period, subjects were randomly assigned 2:1 to the compound of Formula (I) (n=6) or placebo (n=3). Each subject received one dose of placebo and two different doses of the compound of Formula (I) over the course of their participation in all three dosing periods. Subjects were equally randomized to one of 3 possible sequences, namely 1) placebo, active, active, 2) active, placebo, active, and 3) active, active, placebo.

The single ascending dose Phase 1A study was successfully completed with no safety signals seen that were attributable to the compound of Formula (Ia).

### Example 12

### Evaluation of Efficacy of a Compound of Formula (Ia) in an Allergic Asthma Model

### Materials and Methods

Animal Preparation:

Compounds were tested in the sheep model of experimental asthma reported in Abraham W M, Asthma & Rhinitis, 2000: 1205-1227 incorporated herein by reference in its entirety. All animals were demonstrated to have both early and late airway responses to inhalation challenge with Ascaris suum antigen.

Measurement of Airway Mechanics:

The unsedated sheep were restrained in a cart in the prone position with their heads immobilized. After topical anesthesia of the nasal passages with 2% lidocaine solution, a balloon catheter was advanced through one nostril into the lower esophagus. The sheep were intubated with a cuffed endotracheal tube through the other nostril. Pleural pressure was estimated with the esophageal balloon catheter. Lateral pressure in the trachea was measured with a sidehole catheter (inner dimension, 2.5 mm) advanced through and positioned distal to the tip of the endotracheal tube. Transpulmonary pressure, the difference between tracheal and pleural pressure, was measured with a differential pressure transducer catheter system. For the measurement of pulmonary resistance (R_{L}), the proximal end of the endotracheal tube was connected to a pneumotachograph. The signals of flow and transpulmonary pressure were sent to a computer for on-line calculation of R_{L} from transpulmonary pressure, respiratory volume (obtained by digital integration) and flow. Analysis of 5-10 breaths was used for the determination of R_{L} in L×cm H₂O/L/S.

Aerosol Delivery Systems:

Aerosols of Ascaris suum extract (diluted 20:1 with phosphate buffered saline; 82,000 PNU/ml) were generated using a disposable medical nebulizer (Raindrop^{R}, Puritan Bennett). The output from the nebulizer was directed into a plastic t-piece, one end of which was connected to the inspiratory port of a Harvard respirator. To better control aerosol delivery, a dosimeter consisting of a solenoid valve and a source of compressed air (20 psi) was activated at the beginning of the inspiratory cycle of the Harvard respirator system for 1 second. The aerosol was delivered at a tidal volume of 500 ml and a rate of 20 breaths per minute. Carbachol aerosols were also generated with the nebulizer system described above.

Pulmonary Resistance Response Data:

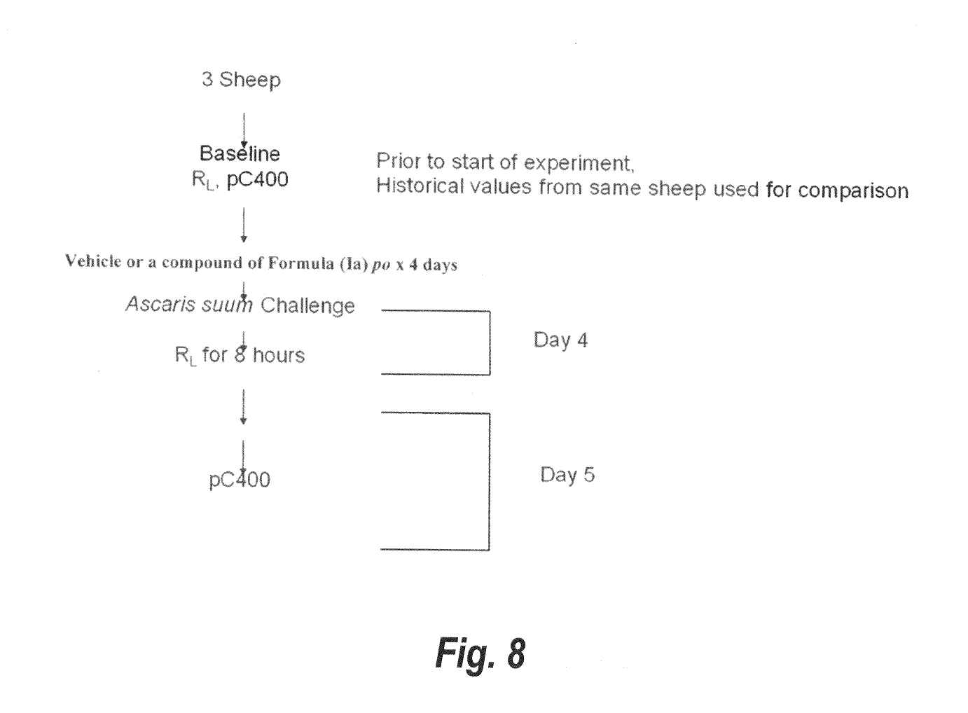

Pulmonary resistance data for the compound of Formula (Ia) was collected according to the flow chart shown in FIG. 8. Baseline concentration response curves to aerosol Carbachol were obtained 1 to 3 days before commencement of drug/vehicle treatment. Referring to FIG. 8, sheep (n=3/group) received daily administration of the compound of Formula (Ia) at 3, 5, or 10 mg/kg per os (PO) or vehicle at 5 mL/kg PO for 4 consecutive days. Sheep were fasted each night prior to dosing the following morning. On the challenge day, which was the 4^{th} consecutive day of administration, sheep were treated with the compound of Formula (Ia) or Vehicle, orally, 2 hours prior to antigen challenge. Baseline values of pulmonary resistance (R_{L}) were obtained within 30 minutes of treatment and then re-measured 30 minutes before antigen challenge. Measurements of R_{L} were obtained immediately after the sheep were challenged with Ascaris suum antigen, hourly from 1-6 hours after challenge and on the half-hour from 6½ to 8 hours after challenge. Determination of the 24 hour post-challenge concentration response curve was performed.

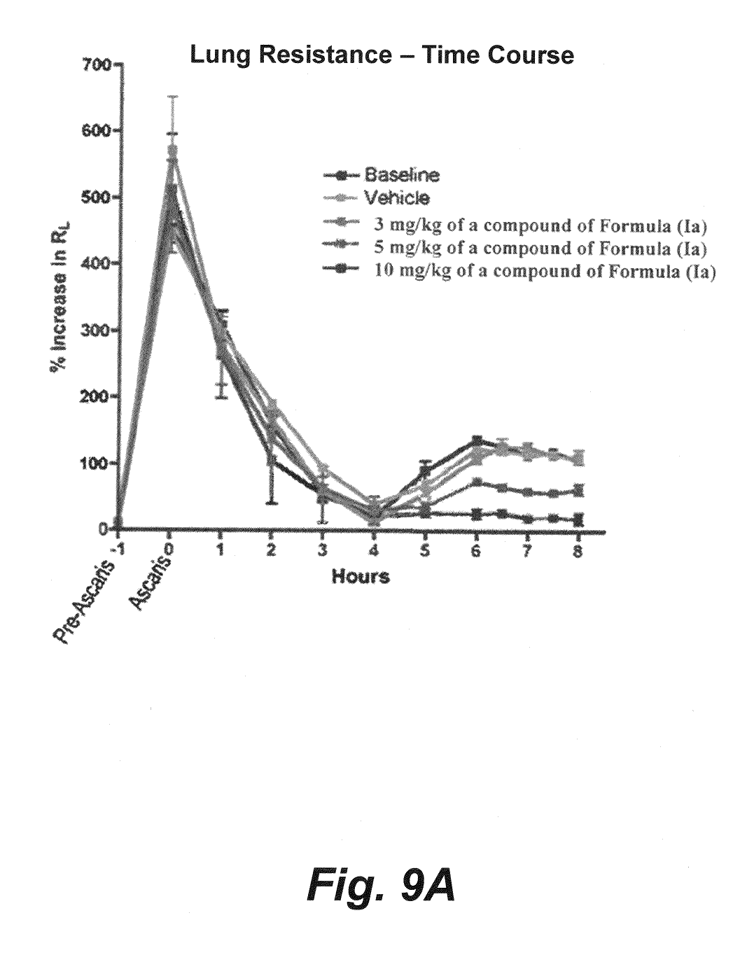

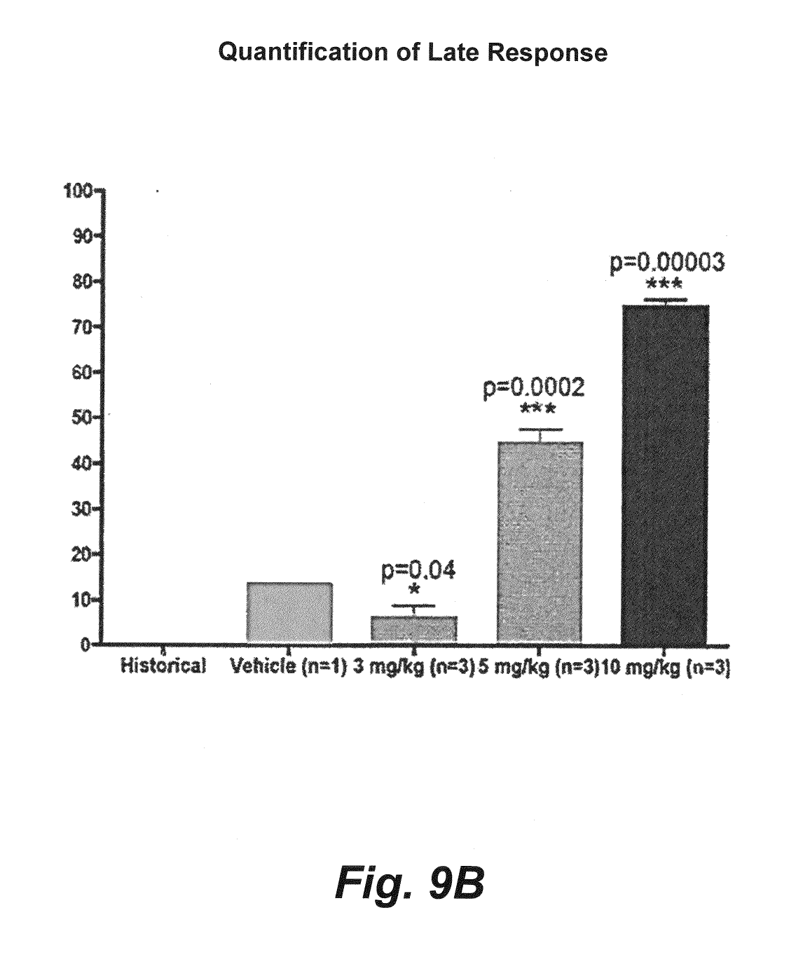

FIG. 9A shows data calculated from the measurement of pulmonary resistance (R_{L}) as a function of time (hours). FIG. 9B shows the quantification of the late phase response to the compound of Formula (Ia) seen in FIG. 9A. Referring to FIG. 9A, lung resistance peaked soon after Ascaris suum antigen was administered, and then R_{L} decreased until time 4 hours. Late phase increase in R_{L} was observed starting at 4 hours post-antigen challenge in both the baseline and vehicle. Similar effects were also seen at a dose of 3 mg/kg of the compound of Formula (Ia). However, doses of 5 and 10 mg/kg were effective at lowering pulmonary resistance in the late phase of the time course. At a dose of 5 mg/kg of the compound of Formula (Ia), the 5 mg/kg dosage showed an approximate 45% inhibition, while at a dose of 10 mg/kg, % inhibition was quantified as approximately 75% (FIG. 9B).

Airway Hyper-Responsiveness (PC₄₀₀) Data:

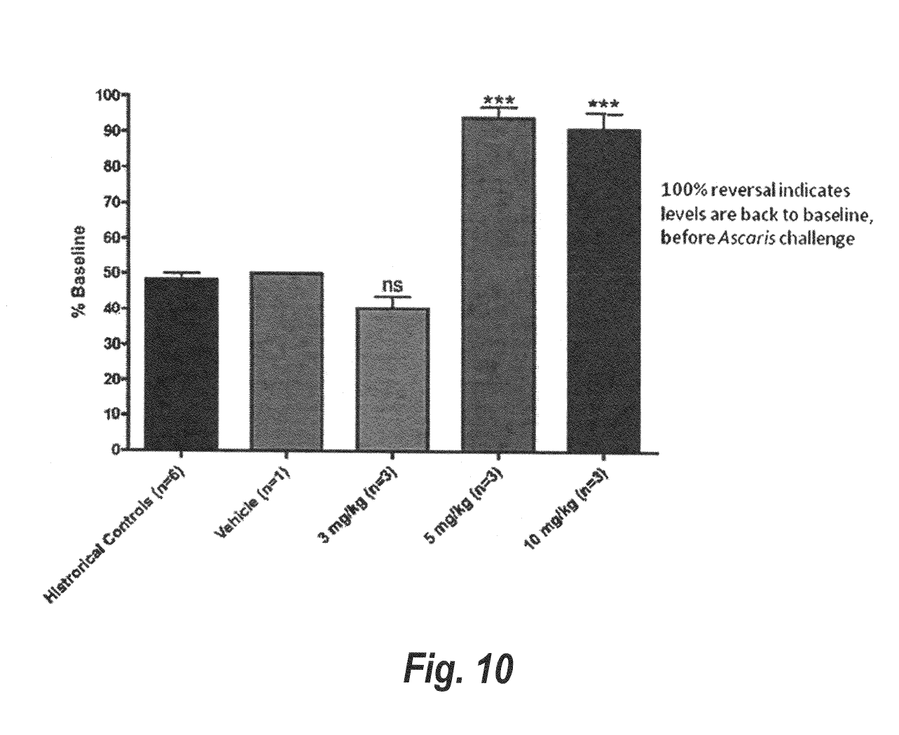

Measurements of R_{L} were repeated immediately after inhalation of buffer and after each administration of 10 breaths of increasing concentrations of Carbachol solution (0.25%, 0.5%, 1.0%, 2.0% and 4.0% w/v). To assess airway responsiveness, the cumulative Carbachol dose in breath units (BU) that increased R_{L} 400% over the post-buffer value (i.e. PC₄₀₀) was calculated from the dose response curve (FIG. 10). One breath unit is defined as one breath of a 1% w/v Carbachol solution. Referring to FIG. 10, doses of 5 and 10 mg/kg of a compound of Formula (Ia) showed a 100% increase in baseline indicating the R_{L} was back to levels prior to antigen challenge.

### Incorporation by Reference

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for treating asthma, comprising administering by pulmonary or intranasal delivery to a subject in need thereof a pharmaceutical composition comprising a therapeutically effective amount of a compound of Formula (Ia), or a pharmaceutically acceptable salt thereof:

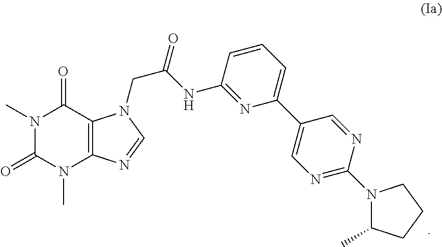 [SMILES: C[C@H]1CCCN1c1ncc(-c2cccc(NC(=O)Cn3cnc4c3c(=O)n(C)c(=O)n4C)n2)cn1]

2. The method of claim 1, wherein the compound of Formula (Ia) is in the form of a hydrochloride salt.

3. The method of claim 1, wherein the pharmaceutical composition is administered by inhalation.

4. The method of claim 1, wherein method is for the treatment of human asthma.

5. The method of claim 4, wherein the human asthma is selected from the group consisting of: cold induced asthma, exercise-induced asthma, allergy-induced asthma, and occupational asthma.

6. The method of claim 1, wherein the pharmaceutical composition is administered as an aerosol.

7. The method of claim 1, wherein the pharmaceutical composition is administered at a dose of about 0.5-25 mg/kg.

8. The method of claim 1, wherein the pharmaceutical composition is administered at a dose of about 5-10 mg/kg.

9. The method of claim 1, wherein the pharmaceutical composition is administered in a unit dosage form that comprises about 200 mg-1600 mg of a compound of Formula (I).

10. The method of claim 1, wherein the pharmaceutical composition is administered in a unit dosage form that comprises about 200 mg, 400 mg, 800 mg, 1200 mg or 1600 mg of a compound of Formula (I).

11. The method of claim 6, wherein the pharmaceutical composition is administered as droplets of about 5 mm or less in diameter.

12. The method of claim 1, wherein the pharmaceutical composition is administered using metered dose aerosol dispenser containing the pharmaceutical composition.

13. The method of claim 1, wherein the pharmaceutical composition is administered using a metered dose inhaler, a dry powder inhaler or an air-jet nebulizer.

14. The method of claim 1, wherein the pharmaceutical composition is administered with a pH in the range of about 4.5 to 5.5.

15. A method of treatment, comprising administering to a subject a pharmaceutical composition indicated for use in patients with a diagnosis of asthma, the pharmaceutical composition comprising a therapeutically effective amount of a compound of Formula (Ia), or a pharmaceutically acceptable salt thereof:

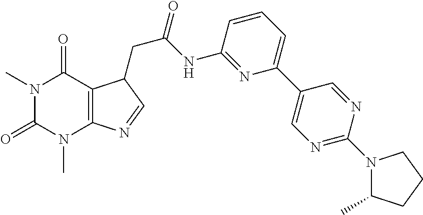 [SMILES: C[C@H]1CCCN1c1ncc(-c2cccc(NC(=O)CC3C=Nc4c3c(=O)n(C)c(=O)n4C)n2)cn1]

16. The method of claim 15, wherein the pharmaceutical composition is administered in a unit dosage form that comprises about 200 mg-1600 mg of a compound of Formula (Ia).

17. The method of claim 15, wherein the compound of Formula (Ia) is in the form of a hydrochloride salt.

18. The method of claim 15, wherein the compound of Formula (Ia) is in the form of a hydrochloride salt and the compound of Formula (Ia) is administered by inhalation.

19. The method of claim 15, wherein the pharmaceutical composition is administered by inhalation or intranasal administration.

20. A dispenser containing a pharmaceutical composition adapted for pulmonary or nasal delivery, the pharmaceutical composition comprising a compound of Formula (Ia), or a pharmaceutically acceptable salt thereof:

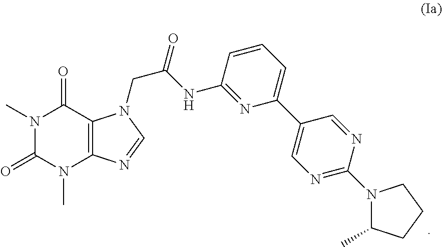 [SMILES: C[C@H]1CCCN1c1ncc(-c2cccc(NC(=O)Cn3cnc4c3c(=O)n(C)c(=O)n4C)n2)cn1]

21. The dispenser of claim 20, wherein the dispenser is a metered dose aerosol dispenser or a dry powder inhalation dispenser.
